(19) United States
(12) Patent Application Publication
(10) Pub. No.: US 2006/0293264 A1
(43) Pub. Date: Dec. 28, 2006

(54) STAT3 decoy oligonucleotides and uses therefor

(76) Inventors: Jennifer R. Grandis, Pittsburgh, PA (US); Daniel E. Johnson, Glenshaw, PA (US); Paul Leong, Pittsburgh, PA (US)

Correspondence Address: JESSE A. HIRSHMAN, ESQ., 1722 MURRAY AVENUE, PITTSBURGH, PA, 15217, US

(21) Appl. No.: 11/188,248

(22) Filed: Jul. 22, 2005

Related U.S. Application Data
(60) Provisional application No. 60/590,747, filed on Jul. 22, 2004.

(51) Int. Cl. A61K 48/00; A61K 33/36; A61K 31/7072; A61K 31/7048; A61K 31/704; A61K 31/525; A61K 31/675; A61K 31/66; A61K 31/337

(52) U.S. Cl. 514/44; 424/649; 514/81; 514/700; 514/34; 514/49; 514/27; 514/109; 514/449; 514/283; 424/623; 514/410; 514/251

(57) ABSTRACT

A composition is provided that is useful in treating cancers in which STAT3 is activated, such as squamous cell carcinomas including squamous cell carcinoma of the head and neck. The composition comprises an effective amount of a STAT3 decoy and a pharmaceutically acceptable carrier. Also provided are methods of treating such cancers and methods of modulating STAT3 transcriptional activation in a cell.

### CROSS REFERENCE TO RELATED APPLICATIONS

This application claims the benefit under 35 U.S.C. §119(e) to U.S. Provisional Patent Application No. 60/590,747, filed Jul. 22, 2004, which is incorporated herein by reference in its entirety.

### STATEMENT REGARDING FEDERAL FUNDING

This invention was made with government support under Grant No. R01CA77308-01, awarded by the National Institutes of Health. The government has certain rights in this invention.

### BACKGROUND

STAT3 oligonucleotide decoys are described herein along with compositions comprising STAT3 oligonucleotide decoys and methods of their use.

The family of Signal Transducers and Activators of Transcription (STATs) play a central role in signaling by numerous cytokines, polypeptide growth factors, and oncoproteins. STATs were initially described in the context of regulating physiologic cell signaling contributing to such diverse processes as differentiation, proliferation, and apoptosis. An increasing number of studies have implicated STAT activation, particularly STAT3, in transformation and tumor progression. Constitutive activation of STAT3 has been detected in many hematopoietic and solid malignancies, including multiple myeloma, leukemias, lymphomas, mycosis fungoides, as well as carcinomas of the prostate, breast, lung, pancreas, ovary and head and neck (Garcia, R., et al., Oncogene, 20: 2499-2513., 2001; Gouilleux-Gruart, V., et al. Blood, 87. 1692-1697., 1996; Grandis, J. R., et al. Proc Natl Acad Sci USA, 97: 4227-4232., 2000; Huang, M., et al. Gynecol Oncol, 79: 67-73, 2000; and Bowman, T., et al. Oncogene, 19: 2474-2488., 2000). Upon activation, STAT proteins dimerize and translocate to the nucleus where they regulate gene expression by binding to specific DNA-response elements (Darnell, J. E., Jr., Science, 277: 1630-1635., 1997). To directly address the role of STAT3 as an oncogene, a constitutively active mutant of STAT3 was generated (STAT3C) and shown to induce transformation of fibroblasts and tumor formation in nude mice (Yu, C. L., et al., Science, 269: 81-83., 1995 and Bromberg, J. F., et al., Cell, 98: 295-303., 1999). In addition to being a point of convergence for numerous oncogenic signaling pathways, STAT3 also participates in cell growth and survival. One of the first indications that STAT3 signaling contributes to malignancy, at least in part by preventing apoptosis, came from studies showing that increased expression of the anti-apoptotic Bcl-2-family gene bcl-x_{L} is dependent on constitutively activated STAT3 in multiple-myeloma cells (Catlett-Falcone, R., et al., Curr. Opin. Oncol. (1999) 11:490-496). Inhibition of STAT3 signaling blocked the expression of Bcl-x_{L} in these tumor cells and sensitized them to FAS-mediated apoptosis (Catlett-Falcone, R., Curr. Opin. Oncol. (1999) 11:490-496). Consistent with these findings, STAT3 activation has been shown to regulate Bcl-x_{L} expression and apoptosis in a wide range of tumor cells (Grandis, J. et al., Proc Natl Acad Sci U S A, 97: 4227-4232., 2000; Bromberg, J. et al., Cell, 98: 295-303., 1999; and Niu, G., et al., Oncogene (2002) 21:2000-2008).

The association of STAT3 activation with transformation and tumor progression suggests that STAT3 may be an attractive molecular target for cancer therapy. Several strategies have been used to block the action of STAT proteins, including antisense methods, ectopic expression of dominant-negative mutants (Grandis, J. R., et al., Embo J, 15. 3651-3658, 1996; and Li, L. et al., J Biol Chem, 277: 17397-17405, 2002) (11-13), inhibition of upstream kinases (Fry, D. et al., Science, 265: 1093-1095, 1994; Kraker, A. J., et al., Biochem Pharmacol, 60: 885-898, 2000; and Turkson, J., et al., Mol Cell Biol, 19: 7519-7528., 1999), and phosphotyrosyl peptides (Turkson, J., et al., J Biol Chem, 276: 45443-45455, 2001). An alternative approach to target the action of transcription factors, including STAT proteins, involves the use of double-stranded “decoy” oligonucleotides. The double-stranded DNA decoy closely corresponds to the response element within the promoter region of a responsive gene. By achieving a sufficient concentration of decoy in the target cells, the authentic interaction between a transcription factor and its endogenous response element in genomic DNA is impaired, with subsequent modulation of gene expression (U.S. Patent Publication Nos. 20020052333, 20020128217 and 20030186922 and Nabel, E. G., et al., Science, 249: 1285-1288., 1990).

It was previously reported that a transcription factor decoy approach could be used to decrease STAT3 activation and target gene expression in squamous cell carcinomas of the head and neck (SCCHN) in vitro (Leong, P. L., et al., Proc Natl Acad Sci USA, 100: 4138-4143, 2003). However, the usefulness of STAT3 decoy in treating cancer in vivo, was not evaluated.

### SUMMARY

The therapeutic potential and mechanisms of the STAT3 decoy was evaluated in an animal model of head and neck cancer. Intratumoral administration of the STAT3 decoy abrogated STAT3 activation and target gene expression in vivo. Decreased tumor volumes in the STAT3 decoy treated tumors was accompanied by increased apoptosis. The potential benefit of combining the STAT3 decoy with an anticancer agent also was evaluated. Both in vitro and in vivo experiments demonstrated that the STAT3 decoy delivered in conjunction with cisplatin resulted in increased antitumor effects compared with either treatment alone.

A composition is therefore provided comprising an amount of a STAT3 decoy effective to: reduce growth of a cancer in vivo in which STAT3 is activated; interfere with STAT3 binding to a STAT3 response element in vivo; and/or induce apoptosis in a cancer cell in which STAT3 is activated, when used in combination with a pharmaceutically acceptable carrier. The composition may further comprise an anticancer agent, such as one or more of AG-490; aldesleukin; alemtuzumab; alitretinoin; allopurinol; altretamine; amifostine; An-238; anastrozole; arsenic trioxide; asparaginase; BCG Live; bevacizumab; bexarotene; bleomycin; busulfan; calusterone; capecitabine; capecitabine; carboplatin; carmustine; celecoxib; cetuximab; chlorambucil; cisplatin; cladribine; cyclophosphamide; cyclophosphamide; cytarabine; dactinomycin; darbepoetin alfa; daunorubicin; daunorubicin, daunomycin; denileukin diftitox; dexrazoxane; docetaxel; doxorubicin; dromostanolone propionate; Elliott's B Solution; endostatin; epirubicin; epoetin alfa; estramustine; etoposide phosphate; etoposide, VP-16; exemestane; filgrastim; floxuridine; fludarabine; fluorouracil; FTI-277; fulvestrant; gefitinib; gemcitabine; gemcitabine; gemtuzumab ozogamicin; GGTI-298; goserelin acetate; gossypol; hydroxyurea; ibritumomab; idarubicin; idarubicin; ifosfamide; imatinib mesylate; interferon alfa-2a; IL-2; IL-12; interferon alfa-2b; irinotecan; letrozole; leucovorin; levamisole; lomustine; meclorethamine; nitrogen mustard; megestrol acetate; melphalan, L-PAM; mercaptopurine, 6-MP; mesna; methotrexate; methoxsalen; mitomycin C; mitotane; mitoxantrone; nandrolone phenpropionate; nofetumomab; oprelvekin; oxaliplatin; paclitaxel; pamidronate; pegademase; pegaspargase; pegfilgrastim; pentostatin; pentostatin; pipobroman; plicamycin; mithramycin; porfimer sodium; PP2; procarbazine; quinacrine; rasburicase; PC3095; rituximab; sargramostim; streptozocin; talc; tamoxifen; temozolomide; teniposide, VM-26; testolactone; thioguanine, 6-TG; thiotepa; topotecan; toremifene; tositumomab; trastuzumab; tretinoin, ATRA; UO 126; uracil mustard; valrubicin; vinblastine; vincristine; vinorelbine; wortmanin and zoledronate. In one embodiment the anticancer agent is cisplatin. In another embodiment, the anticancer agent is gossypol. The composition may be formulated, without limitation, as a dosage form such as, without limitation: a parenteral dosage form, an intravenous and an intratumor dosage form.

The STAT3 decoy typically, but not exclusively, is a double-stranded oligonucleotide or oligonucleotide analog, such as a phosphorothioate nucleic acid analog. In one typical embodiment, the STAT3 decoy is a double-stranded deoxyribonucleotide or an analog thereof comprising the STAT3 target sequence:

(SEQ ID NO:1)

5′-(N₆)ₙ-CAN₁TTCN₂CN₃TN₄AN₅TC-(N₇)ₘ-3′,

wherein N₁, N₂, N₃, N₄ and N₅ are A, T, G or C, and one, two, three or all of the following conditions are met: N₁ is T; N₂ is C; N₃ is G, N₄ is A and N₅ is A, and N₆ and N₇ are A, T, G or C and n and m are independently 0-50. In another embodiment, the decoy is a double-stranded deoxyribonucleotide or an analog thereof comprising a derivative of the STAT3 target sequence:

(SEQ ID NO:2)

5′-(N₆)ₙ-CATTTCCCGTAAATC-(N₇)ₘ-3′,

in which N₆ and N₇ are A, T, G or C and n and m are independently 0-50, containing a single nucleotide insertion, deletion or substitution within the sequence 5′-CATTTCCCGTAAATC-3′ (SEQ ID NO: 2).

Also provided is a method of 1) reducing growth of a cancer in which STAT3 is activated in a patient, 2) interfering with STAT3 binding to a STAT3 response element in cancer cells of a patient in which STAT3 is activated, and/or 3) inducing apoptosis in cancer cells of a patient in which STAT3 is activated. The method comprises administering to the patient an amount of the above-described composition effective to reduce growth of the cancer in which STAT3 is activated in the patient, interfere with STAT3 binding to a STAT3 response element in cancer cells of the patient in which STAT3 is activated and/or induce apoptosis in cells in which STAT3 is activated in the patient. In one embodiment, the method further includes administering to the patient a second anticancer therapy, such as, without limitation, radiation therapy or treatment with an anticancer agent, such as, without limitation, one or more of the anticancer agents listed above.

Further, a method of decreasing expression of one or more genes under transcriptional control by one or more of a p53 response element, a gamma-interferon activated sequence (GAS) and an Egr-1 (Early Growth Response-1) transcription recognition sequence in a cell is provided. The method comprises contacting the cell with composition comprising an amount of a STAT3 decoy effective to decrease expression of the one or more genes subject to control by one or more of a p53 response element, a gamma-interferon activated sequence (GAS) and an Egr-1 (Early Growth Response-1) transcription recognition sequence in a cell, thereby decreasing expression of the one or more genes subject to control by one or more of a p53 response element, a gamma-interferon activated sequence (GAS) and an Egr-1 (Early Growth Response-1) transcription recognition sequence in the cell. The one or more genes may be one or more of a p53 gene and an Egr-1 gene and an allele or mutant of a p53 or Egr-1 gene.

Lastly, a kit is provided comprising a package, a container within the package; one or more doses of a STAT3 decoy in a pharmaceutically acceptable carrier within the container; and a label or package insert providing an indication of the use for the one or more doses in treatment of a cancer. The use can be, without limitation, one of: 1) reducing growth of a cancer in which STAT3 is activated in a patient, 2) interfering with STAT3 binding to a STAT3 response element in cancer cells of a patient in which STAT3 is activated, 3) inducing apoptosis in cancer cells of a patient in which STAT3 is activated, and/or treating a cancer, such as a cancer in which STAT3 is activated, including without limitation, a squamous cell carcinoma or a squamous cell carcinoma of the head and neck.

### BRIEF DESCRIPTION OF THE DRAWINGS

FIG. 1. SCCHN cells (1×10⁶ of 1483 cells) were inoculated subcutaneously in the right and left flank of 10 athymic nude mice. After 10 days when the tumors were clearly palpable (approximately 2 mm in maximum diameter), the tumor on the left flank was treated with daily injections of the STAT3 decoy (25 μg) and the tumor on the right flank was treated with the mutant control decoy (25 μg) in conjunction with tumor volume determinations. The median (solid lines) tumor volumes are shown. Tumor volumes in the STAT3 decoy treated group were significantly decreased on days 25-35 compared with mutant control decoy therapy (p=0.002).

FIGS. 2A-2C. SCCHN xenografts treated with daily intratumoral inoculation of STAT3 decoy or the mutant control decoy were harvested at the end of treatment (25 treatments) and stained for apoptotic cells by TUNEL. Representative staining for DNA fragmentation as well as the cumulative results from 20 tumors are shown (p=0.00038).

FIG. 3A-3H. STAT3 decoy decreases STAT3 activation and target gene expression in vivo. Ten mice bearing SCCHN xenografts were treated with daily injections of STAT3 decoy (tumor on left flank) or mutant control decoy (tumor on right flank) for a total of 25 treatments. Tumors were harvested and analyzed for (FIGS. 3A and 3B) STAT3 activation by EMSA (p=0.02), (FIGS. 3C and 3D) STAT5 activation by EMSA (p>0.05), (FIGS. 3E and 3F) Bcl-x_{L} expression by immunoblotting (p=0.0002) or (FIGS. 3G and 3H) Cyclin D₁ expression by immunoblotting (p=0.0002). Bar graphs represent the cumulative results from 20 tumors analyzed.

FIG. 4. Increased apoptosis of STAT3 decoy plus cisplatin in vitro. SCCHN cells (1483) were treated with mutant control decoy (25 μM, 6 days), or STAT3 decoy alone (25 μM, 6 days), or cisplatin alone (20 μM, 24 hr), or STAT3 decoy (25 μM, 6 days) plus cisplatin (20 μM, 24 hr) followed by an Annexin 5-Cy3 apoptosis assay and fluorescence microscopy (40×) (p=00016).

FIGS. 5A-5D. Enhanced effects of STAT3 decoy plus cisplatin. SCCHN cells (1483) were treated with mutant control decoy (25 μM, 6 days), or STAT3 decoy alone (25 μM, 6 days), or cisplatin alone (20 μM, 24 hr), or STAT3 decoy (25 μM, 6 days) plus cisplatin (20 μM, 24 hr). The effects of the STAT3 decoy plus cisplatin on STAT3 target gene expression (FIGS. 5A and 5B) Bcl-x_{L} and (FIGS. 5C and 5D) Cyclin D1 were examined. Bar graphs represent cumulative data from 3 experiments (p<0.0001).

FIGS. 6A-6J. STAT3 decoy in combination with cisplatin inhibits SCCHN growth, induces apoptosis and inhibits STAT3 target gene expression in vivo. (FIG. 6A) SCCHN cells were inoculated subcutaneously in the right and left flank of athymic nude mice. After 10 days when the tumors were clearly palpable (approximately 2 mm in maximum diameter), mice were randomly assigned to treatment groups (STAT3 decoy, mutant control decoy, ciplatin alone, cisplatin plus STAT3 decoy, cisplatin plus mutant control decoy). There were 6-8 mice in each treatment group. Cisplatin (5 mg/kg) was injected introperitoneally, and intratumoral injection of decoy (25 mg/kg) in a volume of 50 μl was delivered daily. Tumor volumes were measured all over the course. Ten days after initiating therapy in established tumors, the group receiving STAT3 decoy combined with cisplatin were growth inhibited compared with STAT3 decoy combined with mutant control decoy or cisplatin alone (p=0.02), an effect that persisted throughout treatment (FIG. 6A). (FIG. 6B) SCCHN xenografts were harvested at the end of treatment and stained for apoptotic cells by TUNEL. Cumulative results are shown (p=0.002). STAT3 decoy in combination with cisplatin decreases STAT3 target gene expression in vivo. Tumors were harvested and analyzed for (FIGS. 6C and 6D) VEGF expression (p=0.0004), (FIGS. 6E and 6F) BclC-x_{L} expression (p=0.0001), (FIGS. 6G and 6H) Cyclin D₁ expression (p=0.00038), or (FIGS. 6I and 6J) PCNA expression by immunoblotting (p=0.00054). Bar graphs represent the cumulative results from the all tumors analyzed.

FIG. 7. Gossypol Dose Response Curve for PCI-15B cells.

FIG. 8. Graph showing the inhibition of expression from a p53-responsive element-luciferase reporter gene by a STAT3 decoy.

FIG. 9. Graph showing the inhibition of expression from a GAS-responsive element-luciferase reporter gene by a STAT3 decoy.

FIG. 10. Autoradiograph of an EMSA showing the binding of STAT 1 and STAT3 to a STAT3 decoy.

### DETAILED DESCRIPTION

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within these ranges. Also, the disclosure of these ranges is intended as a continuous range including every value between the minimum and maximum values.

Cumulative evidence supports a role for aberrant STAT3 activation in transformation and tumor progression. Increased STAT3 activation in head and neck carcinogenesis has previously been demonstrated, where STAT3 activity contributes to the loss of growth control by an antiapoptotic mechanism (Grandis, J. R., et al., Laryngoscope, 110: 868-874., 2000). Targeting STAT3 with antisense oligonucleotides or dominant-negative mutants has resulted in apoptosis and modulation of STAT3 regulated genes in several cancer-derived cell lines including multiple myeloma, melanoma, mycosis fungoides, and SCCHN (Grandis, J. R., et al., Proc Natl Acad Sci USA, 97: 4227-4232., 2000; Catlett-Falcone, R., et al., Immunity, 10. 105-115., 1999.; Niu, G., et al., Cancer Res, 59: 5059-5063., 1999; and Nielsen, M., et al., Leukemia, 13: 735-738, 1999). Evidence is provided herein that STAT3 activation can be targeted in vivo using a transcription factor decoy approach with antitumor effects. The examples below show use of a double-stranded oligomer with phosphorothioate modifications, inhibiting tumor growth, increasing apoptosis and abrogating STAT3 target genes in an in vivo model of head and neck cancer. Furthermore, the addition of cisplatin to the STAT3 decoy increased growth inhibitory effects in vitro and antitumor effects in vitro and in vivo. These results demonstrate the usefulness of the STAT3 decoy as a therapeutic, either alone, or in combination with an anticancer agent for head and neck cancer patients.

One feature of an ideal cancer therapy is that it would specifically target tumor cells, without toxicity to normal cells. STAT3-null mice show an embryonic-lethal phenotype (Takeda, T., et al., J. Endocrinol, 153: R1-3, 1997), which indicates a requirement for STAT3 signaling during early development. However, many studies that involve tissue-specific ablation of STAT3 have shown that STAT3-null non-tumor cells proliferate and survive well in vivo and in vitro. These include STAT3 macrophages, neutrophils, mammary cells, bone-marrow progenitors, keratinocytes and mouse embryonic fibroblasts (Levy, D. E. et al., Nat Rev Mol Cell Biol, 3: 651-662, 2002 and Akira, S. Oncogene, 19: 2607-2611, 2000). Numerous studies have shown that blocking constitutively activated STAT3 leads to inhibition of tumor cell growth and apoptosis of tumor cells (Turkson, J., et al., J Biol Chem, 276: 45443-45455, 2001; Leong, P. L., et al., Proc Natl Acad Sci USA, 100: 4138-4143, 2003 and Bowman, T., Proc Natl Acad Sci USA, 98: 7319-7324., 2001). The STAT3 decoy described herein was previously reported to lack toxicity to normal mucosal epithelial cells despite incorporation of the decoy into these cells (Leong, P. L. et al., Proc Natl Acad Sci USA, 100: 4138-4143, 2003). This selective inhibition might reflect an irreversible dependence of tumor cells on high levels of activated STAT3 for growth and survival, whereas normal cells might be able to withstand lower levels of STAT3 activity, or use alternative pathways for growth and survival.

STAT3 regulates cell growth and survival, at least in part, by preventing apoptosis through increased expression of the anti-apoptotic gene bcl-x_{L} (Bromberg, J. F., et al., Cell, 98: 295-303., 1999). Inhibition of STAT3 signaling by various means decreased expression of Bcl-XL in tumor cells and sensitized them to FAS-mediated apoptosis (Grandis, J. R., et al., Proc Natl Acad Sci USA, 97: 4227-4232., 2000; Catlett-Falcone, R., et al., Immunity, 10: 105-115., 1999.; Niu, G., et al., Cancer Res, 59: 5059-5063., 1999; and Nielsen, M., et al., Leukemia, 13: 735-738, 1999). In addition, many growth-factor signaling pathways are known to regulate cell proliferation by enhancing the activity of cyclins, contributing to accelerated cell-cycle progression. Constitutive activation of STAT3 is associated with cyclin D1 upregulation (Bromberg, J. et al., Cell, 98:295-303, 1999). In addition to cell proliferation and cell survival, angiogenesis is also required for tumor development. Most tumors cannot sustain their growth unless they are supplied with oxygen and nutrients from newly formed blood vessels. One of the most potent angiogenesis-inducing signals is vascular endothelial growth factor (VEGF). VEGF is usually produced by cancer cells in higher level than their normal counterparts. VEGF binds to a transmembrane receptor tyrosine kinase on endothelial cells, activates endothelial-cell migration and proliferation to form new blood vessels. STAT3 has been shown to be a direct transcriptional activator of the VEGF gene (Niu, G., et al., Oncogene, 21: 2000-2008, 2002 and Wei, D., et al., Oncogene, 22: 319-329, 2003). Transfection of cells with the constitutively activated mutant STAT3C is sufficient to increase VEGF expression and induce angiogenesis in vivo (Niu, G., et al., Oncogene 21:2000-2008, 2002). Blocking STAT3 signaling has been shown to inhibit SRC and IL-6-induced VEGF upregulation (Niu, G., et al., Oncogene, 21: 2000-2008, 2002 and Wei, D. et al., Oncogene, 22: 319-329, 2003), and might therefore also abrogate the induction of VEGF by other tyrosine-kinase pathways that lie upstream of STAT3.

The incorporation of the STAT3 decoy into SCCHN cells in vitro was previously assessed using fluorescence labeled flow cytometry and found that the decoy was detected in a high percentage (>90%) of the cells examined. As shown in the examples below, a STAT3 decoy demonstrated marked and reproducible effects on STAT3-mediated growth pathways in vivo.

Transcription factor decoys recently have emerged as potential therapeutic tools for clinical application. Often transcription factors are necessary for cell viability so that stable expression of a dominant-negative transcription factor mutant is often unsuccessful. Use of transcription factor decoys has facilitated the study of transcription factors and their role in oncogenesis. Because transcription factors can recognize their relatively short binding sequences even in the absence of surrounding genomic DNA, short, radiolabeled oligodeoxynucleotides bearing consensus binding sites can serve as probes in electrophoretic mobility shift assays, which identify and quantify transcription factor binding activity in nuclear extracts. More recently, oligodeoxynucleotides bearing the consensus binding sequence of a specific transcription factor have been explored as tools for manipulating gene expression in living cells (Mann, M. J. et al., J Clin Invest, 106: 1071-1075, 2000). This strategy involves the intracellular delivery of such “decoy” oligodeoxynucleotides, which are then recognized and bound by the target factor. Occupation of the transcription factor's DNA-binding site by the decoy renders the protein incapable of subsequently binding to the promoter regions of target genes. Bielinska et al. first described the use of such decoys as a tool for investigating the role of transcription factor activity in cell culture systems (Bielinska, A., et al., Science, 250. 997-1000, 1990). Decoys can also be devised as therapeutic agents, either to inhibit the expression of genes that are transactivated by the factor in question, or to upregulate genes that are transcriptionally suppressed by the binding of a factor. Kawamura et al. examined the role of NF-_{κ}B transactivation in tumor-induced cachexia in mice and found that intratumoral injection of NF-_{κ}B decoy oligodeoxynucleotide into colonic adenocarcinomas decreased food intake, body weight, and muscle mass (Kawamura, I., et al., Gene Ther, 6: 91-97, 1999). Decoy oligodeoxynucleotides also offer a means to specifically inhibit other transcription factors in living cells, both for basic research into the molecular pathways involving these factors (Lim, R., et al., J Neurochem, 74: 596-602, 2000; Takeuchi, S., et al., Brain Res Mol Brain Res, 74: 208-216, 1999; Bishop-Bailey, D. et al., J Biol Chem, 274: 17042-17048, 1999; Boccaccio, C., et al., Nature, 391: 285-288., 1998; and von Knethen, A., et al., Oncogene, 17: 387-394, 1998) and for novel drug development. Transcription factor decoys have been used to block STAT6 activity, which may be useful in reducing IL-4-induced proliferation of Th cells in allergic diseases (Wang, L. H., et al., Blood, 95: 1249-1257., 2000), as well as the expression of cAMP-response element-binding protein (CREB) in tumor cells (Park, S. H., et al., J Biol Chem, 274: 7421-7430, 1999).

STAT3 has been shown to be markedly elevated and to abrogate apoptosis in head and neck squamous cell carcinomas compared with normal oral mucosa from patients without cancer. A 15-mer STAT3 decoy that closely corresponds to the STAT3 response element within the c-fos promoter was constructed and was shown to abrogate head and neck squamous cell carcinoma growth in vitro in dose-dependent manner (Leong, P. L., et al., Proc Natl Acad Sci USA, 100: 4138-4143, 2003). As shown herein, blocking STAT3 activation using a transcription factor decoy approach decreased tumor growth and STAT3 target gene expression in vivo. Blockade of STAT3 with the STAT3 decoy also induced apoptosis, an effect that was augmented when the STAT3 decoy was combined with cisplatin, both in vitro and in vivo. Multimodality therapy has emerged as the treatment of choice for most patients with solid tumors. Cisplatin has proven efficacy in the treatment of SCCHN and is a component of many combined therapeutic strategies (Mayer, F., et al., Ann Oncol, 14: 825-832, 2003). The potential advantages of combining the STAT3 decoy with a second anticancer therapy include the non-overlapping mechanisms and toxicities as well as the potential to reduce the dose of chemotherapy without abrogating antitumor effects. These results suggest that a transcription factor decoy therapeutic approach may be used to target STAT3 in cancers that demonstrate increased STAT3 activation including SCCHN.

In Example 5, below, the STAT3 decoy is shown to have unexpectedly broad impact on the expression not only of STAT3, but on expression facilitated by enhancers and/or transcriptional elements of p53, GAS and Egr-1. The decoy further appears to interfere with STAT1 function. As such, the decoy may find use in treating cancers other than those in which STAT3 is activated. The STAT3 decoy is shown to be useful in decreasing expression of genes under transcriptional control of a p53 response element, a gamma-interferon activated sequence (GAS) and/or an Egr-1 (Early Growth Response-1) transcription recognition sequence in a cell. In one embodiment, the cell may be contacted with composition comprising an amount of a STAT3 decoy effective to decrease expression of one or more genes subject to control by one or more of a p53 response element, a gamma-interferon activated sequence (GAS) and an Egr-1 (Early Growth Response-1) transcription recognition sequence in a cell, thereby decreasing expression of the one or more genes.

By “expression” it is meant the overall flow of information from a gene (without limitation, a functional genetic unit for producing a gene product, typically encoded on DNA or RNA, for some viruses, and comprising a transcriptional promoter, and other cis-acting elements, such as response elements and/or enhancers, an expressed sequence that typically encodes a protein (open-reading frame or ORF) or functional/structural RNA, and a polyadenylation sequence), to produce a gene product (typically a protein, optionally post-translationally modified or a functional/structural RNA) and a transcription termination (polyA) sequence). By “expression of genes under transcriptional control of,” or alternately “subject to control by,” a designated sequence, it is meant gene expression from a gene containing the designated sequence operably linked (functionally attached, typically in cis) to the gene. The designated sequence may be all or part of the transcriptional elements (without limitation, promoters, enhancers and response elements), and may wholly or partially regulate and/or affect transcription of a gene. As a non-limiting example, a gene under transcriptional control of a p53 response element may be wild-type (wt) p53, a p53 allele or mutant, or a recombinant construct, such as the p53-luciferase construct described in Example 5, below.

As used herein a “STAT3 decoy” comprises a double-stranded deoxyribonucleic acid (DNA) or an analog thereof to which STAT3 binds, and which effectively interferes with binding of activated STAT3 to its target DNA sequences in a gene, thereby modulating (changing, altering or otherwise affecting) the effect of activated STAT3 on expression of the gene. A STAT3 decoy can contain any effective sequence, but is defined by its ability to specifically bind STAT3 and to interfere with the binding of STAT3 with its target DNA sequence. As such a STAT3 decoy contains a “STAT3 target sequence”, namely a sequence to which STAT3 binds. For purposes herein, a candidate STAT3 decoy may be tested for its binding affinity and target specificity by electrophoretic mobility shift assay, by binding with STAT3 and by effectively competing with binding of STAT3 to double-stranded DNA comprising a STAT3 target sequence, for example, and without limitation,
5′-AGCTTGTCGACATTTCCCGTAAATCGTCGAG-3′ (SEQ ID NO: 3), and/or
   5′-CAGTTCCCTTAAATC-3′ (SEQ ID NO: 4).

In one embodiment, the STAT3 decoys comprise a double-stranded DNA or an analog thereof comprising the STAT3 target sequence:

5′-(N₆)ₙ-CAN₁TTCN₂CN₃TN₄AN₅TC-(N₇)ₘ-3′,

wherein N₁, N₂, N₃, N₄ and N₅ are A, T, G or C, and one, two, three or all of the following conditions are met: N₁, is T; N₂ is C; N₃ is G, N₄ is A and N₅ is A, and N₆ and N₇ are A, T, G or C and n and m are independently 0-50. In one embodiment, N₂ is a pyrimidine. In a further embodiment, The STAT3 decoy comprises a double-stranded DNA or an analog thereof comprising a derivative of the STAT3 target sequence:

(SEQ ID NO:2)

5′-(N₆)ₙ-CATTTCCCGTAAATC-(N₇)ₘ-3′,

in which N₆ and N₇ are A, T, G or C and n and m are independently 0-50, containing a single nucleotide insertion, deletion or substitution within the sequence 5′-CATTTCCCGTAAATC-3′ (SEQ ID NO: 2). By the phrase “containing a single nucleotide insertion, deletion or substituted within the sequence 5′-CATTTCCCGTAAATC-3′ (SEQ ID NO: 2)” it is meant that any one of the listed bases may be deleted or substituted, or a nucleotide can be inserted in any place between any of the listed nucleotides. In many instances, two or more nucleotides may be inserted, deleted or substituted within the STAT3 target sequence:

(SEQ ID NO:2)

5′-CATTTCCCGTAAATC-3′,

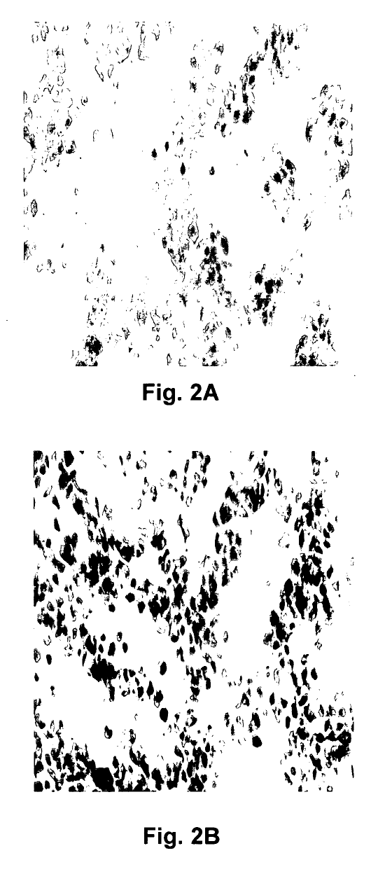

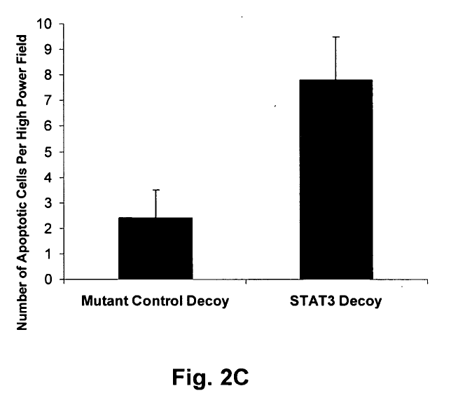

to produce an effective STAT3 decoy (see Table 1). STAT3 decoy consensus sequences and mutants thereof are described herein. Wagner et al. also provides mutational analysis and a consensus sequence for the SIF/STAT3 binding domain, Wagner, B. J., et al., EMBO J. 9(13):4477-4484 (1990)(see, FIG. 2A). The STAT3 decoy sequence can be repeated two or more times in the STAT3 decoy and/or can be concatamerized or otherwise combined with a second, different decoy sequence.

In its most typical embodiment, the STAT3 decoy comprises a double-stranded DNA. As used herein, the term “oligonucleotide” is a double-stranded oligodeoxyribonucleotide. There is no strict size limit to an “oligonucleotide” as defined herein, only that the oligonucleotide can pass into a target cell, by itself or with the assistance of a cell permeation enhancer such as a liposome composition or a peptide transduction domain, for example, TAT (Fischer, P. M. et al., Bioconjugate Chemistry 12(6):825-841 (2001) and Tung, C. H. et al., Bioconjugate Chemistry 11(5):605-618 (2000)), and provides sufficient sequence information to act as a STAT3 decoy. As such, an oligonucleotide typically ranges from 5 to 100 bases. As an example, certain specific oligonucleotides described in the embodiments of the examples, below, are 15 bases in length.

As used herein, an “oligonucleotide analog” and “nucleic acid analog” is a nucleic acid, or a nucleic acid substitute, other than a linear, double-stranded DNA, that is a functional analog of a double-stranded DNA, which, in the context of the present disclosure is an effective STAT3 decoy as determined by, for example and without limitation, the electrophoretic mobility shift assays described herein. Functional analogs are compounds that are suitable for use as STAT3 decoys and therefore have adequate sequence specificity and ability to bind to STAT3 and interfere with the binding of STAT3 with its target DNA sequence. Examples of oligonucleotide or nucleic acid analogs include, without limitation: double-stranded RNA, single-stranded DNA and single-stranded RNA. The DNA analog may be a double-stranded oligonucleotide containing base or backbone chemical modifications that render it less sensitive to degradation when used in vivo. Examples of such modifications include, without limitation, phosphorothioation and methylphosphonation. Single-stranded RNA or DNA may contain secondary structures, creating double-stranded portions containing the decoy sequences. Other structures, such as, without limitation, a circular dumbbell decoy oligonucleotide structure (see, for example, Ahn, J. D., et al., Circ. Res. 90:1325-1332 (2002)) may be used as a STAT3 decoy. DNA analogs include nucleic acid compositions containing chemical modifications including, without limitation, 5′ and 3′ modifications, backbone modifications and derivatized bases that protect the DNA analog from degradation and/or facilitate entry of the oligonucleotide analog into the target cell.

Non-limiting examples of such modifications include: partial or total phosphorothioation; partial or total methylphosphonation; conjugation to a protein/peptide transduction domain, such as TAT; conjugation to cancer cell-targeting peptides, such as ligands of surface proteins expressed or overexpressed on the surface of a target cancer cell, such as without limitation, Epidermal Growth Factor Receptor (see, Phillips, P. C., et al., Cancer Res. 54(4):1008-15 (1994)); methylation; conjugation to tumor-targeting ligands, such as antibodies, folate or iron; cyclization; dumbbell structure and general chemical modification, that is, substitution of one chemical group for another. As an example of a general chemical modification, one group, such as an H, can be substituted with any saturated or unsaturated hydrocarbon group, including lower alkyl (C₁-C₆), lipid, and polymer (for example, PEG) groups. Examples of modified nucleic acids are provided, without limitation, in U.S. Pat. Nos. 6,653,458, 6,727,044, 6,743,909, 6,753,423 and 6,762,169. Alternately, the STAT3 decoy can include nucleotide sequences permitting maintenance of the decoy, either episomally or integrated in the host cell chromosome, in the target cell. Thus, incorporation of one or more STAT3 binding sequences in a plasmid or viral vector can permit a target cell to maintain either transiently or for longer-term the STAT3 decoy as an episome or integrated into a chromosome. Numerous publications and patent documents describe a variety of nucleic acid vectors, plasmids and the like for propagating and maintaining a desired nucleic acid in an episomal or integrated state. In one non-limiting example, a concatamer of a double-stranded DNA STAT3 decoy described herein is inserted between Adeno-Associated Virus (AAV) ITRs according to well-established recombinant methods and is packaged into a recombinant AAV (rAAV) virus particles in AAV capsid proteins. The rAAV particles can then be used to infect the target cancer cells, typically, but not exclusively, by intratumoral infection (see, for example, U.S. Pat. Nos. 5,139,941, 5,436,146, 5,478,745 and 6,548,286). Other viral vectors, such as, without limitation, retroviral vectors, are useful in transferring the STAT3 decoy into target cells.

The STAT3 decoy is delivered to a patient in a dosage form comprising the STAT3 decoy and a pharmaceutically acceptable carrier. A “carrier” includes as a class any compound or composition useful in facilitating storage, stability, administration, cell targeting and/or delivery of the STAT3 decoy to a target cell or cell population, including, without limitation, suitable vehicles, diluents, solvents, excipients, pH modifiers, salts, colorants, flavorings, rheology modifiers, lubricants, coatings, fillers, antifoaming agents, erodeable polymers, hydrogels, surfactants, emulsifiers, adjuvants, preservatives, phospholipids, fatty acids, mono-di- and tri-glycerides and derivates thereof, waxes, oils and water. In one embodiment, the STAT3 decoy is suspended in water (USP) for delivery in vivo. Pharmaceutically acceptable salts, buffers or buffer systems, including, without limitation, saline, phosphate buffer or phosphate buffered saline (PBS) may be included in the dosage form. Vehicles having the ability to facilitate delivery of nucleic acids and/or nucleic acid analogs to a cell in vivo may be utilized to facilitate delivery of the decoy to the target cells. One non-limiting example of such a vehicle is a cationic liposome system, for example and without limitation as shown in U.S. Pat. Nos. 6,656,498, 6,696,038 and 6,749,863. Additional vehicles having the ability to facilitate delivery of nucleic acids and/or nucleic acid analogs to a cell in vivo, such as the AAV and retroviral vehicles described above, are suited for use in a STAT3 decoy-containing dosage form.

In one embodiment, the STAT3 decoy is delivered intratumorally, which includes delivery internal to a tumor and/or immediately adjacent to a tumor or a cancer cell such that the decoy diffuses to contact the tumor or cancer cell. The STAT3 decoy also may be administered locally, regionally or systematically as desired, for example and without limitation: intravenously, intramuscularly, subcutaneously, dermally, subdermally, intraperitoneally, transdermally, iontophoretically and trans-mucosally. Non-limiting examples of devices useful in delivering the STAT3-containing dosage from to a patient include needle/syringes, catheters, trocars, stents or projectiles.

Depending on the route of administration, varying amounts of the STAT3 decoy will be necessary. Although certain threshold amounts of STAT3 decoy needs to be delivered by any given dosage form by any given route, each dosage form has differing ability to deliver the decoy to the cancer cells. Typically, intratumoral injection of the STAT3 decoy will require the least amounts of the decoy. Intravenous, or intramuscular systemic delivery typically will require the greatest amounts of decoy. Dosage forms that efficiently deliver the decoy to a cell would require less decoy than those that are less efficient. Further, certain cancers will require less decoy than others. Therefore, it is more critical that an effective amount of the STAT3 decoy to be delivered to a patient in order to achieve a desired therapeutic goal, rather than a fixed dose for every patient. Nevertheless, standard dosage regimens may be developed. For example, and without limitation, for intratumoral delivery of a phosphorothioate STAT3 analog of approximately 15 bases, 1 to 1,000 μg, typically in 0.1 μg and 1.0 μg increments, of decoy in water USP may be injected at the tumor site once daily, every other day, weekly, bi-weekly, monthly, bi-monthly, or otherwise as needed. Depending on the progression of the cancer in the individual patient, one or more intratumoral injections may be needed to ensure sufficient contact of the decoy with the cancer cells. The delivered amounts may range between 1 and 1,000 μg, including, without limitation, 1, 5, 10, 25, 50, 100, 250, 500 and 1000 μg, and even higher or lower, as is effective to reach the desired end point, such as, without limitation: reducing growth of a cancer in which STAT3 is activated in a patient, interfering with STAT3 binding to a STAT3 response element in cancer cells of a patient in which STAT3 is activated, and/or inducing apoptosis in a patient's tumor cells in which STAT3 is activated. The dosage form may contain varying concentrations of the STAT3 analog, depending on the desired amount of decoy to deliver, the effectiveness of the dosage form at delivering the decoy to its target cells, and the overall composition of the dosage form. Typical concentration ranges for the decoy are without limitation, 1 μg/mL to 1,000 μg/mL and increments, typically in 0.1 μg/mL and 1.0 μg/mL increments therebetween, including, without limitation 1, 5, 10, 25, 50, 100, 250, 500 and 1000 μg/mL.

Treatment of a patient with the described STAT3 decoy may be combined with other anti-cancer therapies, such as treatment with an anticancer agent and radiation therapy. These therapies can be administered to a patient according to any effective protocol, though the treatments may be modified to optimize the combination treatment along with the STAT3 decoy. For example, and without limitation, radiation therapy is performed by administering to the patient a suitable radiation dose of a suitable time at any suitable interval according to well-established protocols. Anticancer agents are administered according to typical protocols for the given drug. Non-limiting classes of drugs useful in combination with the STAT3 decoy include: tyrosine kinase inhibitors, such as gefitinib (Iressa™) and imatinib mesylate (Gleevec™), monoclonal antibodies, such as rituximab (Rituxan™) and cetuximab (Erbitux™); angiogenesis inhibitors, such as endostatin; immune modulators, such as interleukin-12 (IL-12) and interleukin-2 (IL-2); non-tyrosine kinase inhibitors, such AG490 JAK2 inhibitor and PP2 src family kinase inhibitor; serine/threonine kinase inhibitors, such as UO126 for MEK1/2, wortmanin for PI3K; farnesyl or geranyl transferase inhibitors, such as FTI-277 and GGTI-298; and G-protein-coupled receptor inhibitors, such as RC3095 for bombesin and An-238 for somatostatin.

Non-limiting examples of anticancer agents include: AG-490; aldesleukin; alemtuzumab; alitretinoin; allopurinol; altretamine; amifostine; An-238; anastrozole; arsenic trioxide; asparaginase; BCG Live (Bacillus Calmette-Guérin); bevazizumab; bexarotene; bleomycin; busulfan; calusterone; capecitabine; capecitabine; carboplatin; carmustine; celecoxib; cetuximab; chlorambucil; cisplatin; cladribine; cyclophosphamide; cyclophosphamide; cytarabine; dactinomycin; darbepoetin alfa; daunorubicin; daunorubicin, daunomycin; denileukin diftitox; dexrazoxane; docetaxel; doxorubicin; dromostanolone propionate; Elliott's B Solution; endostatin; epirubicin; epoetin alfa; estramustine; etoposide phosphate; etoposide, VP-16; exemestane; filgrastim; floxuridine; fludarabine; fluorouracil; FTI-2777; fulvestrant; gefitinib; gemcitabine; gemcitabine; gemtuzumab ozogamicin; GGTI-298; goserelin acetate; gossypol; hydroxyurea; ibritumomab; idarubicin; idarubicin; ifosfamide; imatinib mesylate; interferon alfa-2a; interferon alfa-2b; IL-2; IL-12; irinotecan; letrozole; leucovorin; levamisole; lomustine; meclorethamine; nitrogen mustard; megestrol acetate; melphalan, L-PAM; mercaptopurine, 6-MP; mesna; methotrexate; methoxsalen; mitomycin C; mitotane; mitoxantrone; nandrolone phenpropionate; nofetumomab; oprelvekin; oxaliplatin; paclitaxel; pamidronate; pegademase; pegaspargase; pegfilgrastim; pentostatin; pentostatin; pipobroman; plicamycin; mithramycin; porfimer sodium; PP2; procarbazine; quinacrine; rasburicase; RC3095; rituximab; sargramostim; streptozocin; talc; tamoxifen; temozolomide; teniposide, VM-26; testolactone; thioguanine, 6-TG; thiotepa; topotecan; toremifene; tositumomab; trastuzumab; tretinoin, ATRA; UO126; uracil mustard; valrubicin; vinblastine; vincristine; vinorelbine; wortmanin; and zoledronate. A combined dosage form includes an amount of STAT3 decoy and an amount of an anticancer agent effective to reduce growth of a cancer in which STAT3 is activated in a patient, interfere with STAT3 binding to a STAT3 response element in cancer cells of a patient in which STAT3 is activated, and/or induce apoptosis in cancer cells of a patient in which STAT3 is activated. The combined dosage form can be delivered intratumorally, intraperitoneally or intravenously, as is desired.

In one example, the anticancer agent is gossypol (2,2′-bis-(Formyl-1,6,7-trihydroxy-5-isopropyl-3-methylnaphthalene), a drug refined from cottonseed oil and having purported anticancer effects. Gossypol is a BH3 domain small molecule mimetic that targets Bcl-X^{L}. Studies have shown that gossypol binds to the BH3 domain of Bcl-X_{L} and Bcl-2 to cause apoptosis. Gossypol treatment typically induces DNA fragmentation, PARP cleavage, loss of mitochondrial membrane potential, cytochrome c release, and activation of caspase-3,-8, and -9. Because over-expression of Bcl-X_{L} has been reported in SCCHN and reduced expression is associated with increased response to chemotherapy, the use of molecular approaches that target Bcl-X_{L} represents a potential approach to induce apoptosis in SCCHN. Indeed, (−)-gossypol has been shown to be an effective antitumor treatment in SCCHN (Oliver et al., Clin. Cancer Res., 10(22):7757-63, Nov. 15, 2004), but at relatively high concentrations. Gossypol exists in two optical isomers, the (−)-isomer being associated with contraceptive effects, while the (+)-isomer has been implicated in cardiotoxicity in cattle. In one embodiment, the anticancer agent is (−)-gossypol, a composition, which may contain small or trace amounts, and in any case, pharmacologically-insignificant amounts of the (+)-gossypol isomer. A method of resolving a racemic gossypol acetic acid composition into the (+) and (−) enantiomers is provided in Oliver et al., 2004.

An article of manufacture also is provided comprising a package, a container within the package; one or more doses of a STAT3 decoy in a pharmaceutically acceptable carrier within the container; and a label or package insert providing an indication of the use for the one or more doses in treatment of a cancer comprising cells in which STAT3 is activated. As disclosed herein, “treatment of a cancer comprising cells in which STAT3 is activated” includes, without limitation, one or more of the embodiments of: interfering with STAT3 binding to a STAT3 response element in cancer cells of a patient in which STAT3 is activated; reducing growth of a cancer in which STAT3 is activated in a patient; and inducing apoptosis in cancer cells of a patient in which STAT3 is activated. By the term “reducing growth of a cancer,” it is meant reducing in a patient the average growth rate and/or size of a population of cancer cells, such as a tumor or blood cancer.

The compositions, methods and articles of manufacture described herein are effective in treating cancers in which STAT3 is activated. Without limitation, one class of cancers that belong to this group is the squamous cell carcinomas, which also are known as epitheloid cancers. As used herein a “squamous cell carcinoma” is a cancer arising, at least in part, from a squamous cell population and/or containing, at least in part, a squamous cell population including, without limitation, cancers of the cervix; penis; head and neck, including, without limitation cancers of the oral cavity, salivary glands, paranasal sinuses and nasal cavity, pharynx and larynx; lung; esophageal; skin other than melanoma; vulva and bladder. Non-limiting examples of cancers in which STAT3 is activated are: multiple myeloma; HTLV-1 dependent leukemia; acute myelogenous leukemia (AML); Large granular lymphocyte leukemia; lymphomas, including EBV-related Burkitt's lymphoma, mycosis fingoides, cutaneous T-cell lymphoma, non-Hodgkins lymphoma; anaplastic large-cell lymphoma (ALCL), breast cancer, melanoma, ovarian cancer, lung cancer, pancreatic cancer and prostate cancer (Yu, H. et al., Nat. Rev. Cancer, 2004 Feb., 4(2):97-105).

The following examples are intended to further illustrate the invention, without any intent for the invention to be limited to the specific embodiments described therein.

### EXAMPLES

Cells. For both in vitro and the xenograft studies, the 1483 cell line was used. The 1483 cell line is a well-characterized SCCHN cell line derived from a pharyngeal cancer that can form tumors in athymic nude mice (Wagner et al., EMBO J. 9(13):4477-4484 (1990)). In culture, cells were maintained in supplemented DMEM (Cellgro, Washington, DC) with 10% FBS (GIBCO/BRL, Grand Island, N.Y.), plus 100 units/ml penicillin and 100 units/ml streptomycin (GIBCO/BRL).

STAT3 decoy and mutant control decoys. Phosphorothioated sense and antisense strands of STAT3 decoy and mutant control decoy oligonucleotides were designed and obtained from DNA Synthesis Facility, University of Pittsburgh (Pittsburgh, Pa.) by means of β-cyanothylphysphoramidite chemistry to minimize degradation of the oligonucleotides by endogenous nucleases. The STAT3 decoy sequence, based on the hSIE sequence, was 5′-CATTTCCCGTAAATC-3′ (SEQ ID NO: 2), 3′-GTAAAGGGCATTTAG-5′ (SEQ ID NO: 5) and the mutant control decoy sequence was 5′-CATTTCCCTTAAATC-3′(SEQ ID NO: 6), 3′-GTAAAGGGAATTTAG-5′(SEQ ID NO: 7). Sense and antisense strands were dissolved in Tris-EDTA (pH 8.0) at a concentration of 900-1,200 μM. Each sense-antisense pair was annealed by heating to 90° C. and decreasing the temperature by 5° C. increments every 15 min. After 3 hours, the reaction mixture was held at a base temperature of 4° C.

The STAT3 decoy sequence was systematically derived from the sequence of the c-fos gene shown to be necessary for binding of the sis-inducible factor (SIF) as described in Wagner et al., EMBO J. 9(13):4477-4484 (1990). A longer sequence was initially examined, with the shorter decoy sequence identified in bold:

(SEQ ID NO:3)

5′-AGCTTGTCGACATTTCCCGTAAATCGTCGAG-3′

(SEQ ID NO:8)

3′-TCGAACAGCTGTAAAGGGCATTTAGCAGCTC-5′

Using electrophoretic mobility shift assays (EMSAs) it was found that this longer sequence was effective in blocking STAT3 activation. The decoy sequence was systematically shortened, to the sequence indicated in bold, based on the hypothesis that a smaller decoy would be more likely to enter the cell and demonstrate biologic activity.

Derivative decoys were identified by electrophoretic mobility shift assay, performed as described herein. Table 1 describes a number of STAT3 decoy derivatives along with their relative binding to STAT3.

TABLE 1

Sequences and Relative STAT3-DNA Binding Affinities
of STAT3 Decoy and Mutant Decoys

| | |
|---|---|
| Number of | Relative |
| Base Pair | Binding |
| Mutations | to STAT3 |

| | | |
|---|---|---|
| STAT3 Decoy (hSIE) | 5′-CATTTCCCGTAAATC-3′ | ++++ |
| (SEQ ID NOS:2, 5) | 3′-GTAAAGGGCATTTAG-5′ | |
| SIE | 5′-CAGTTCCCTTAAATC-3′ | ++ |
| (SEQ ID NOS:4, 9) | 3′-GTCAAGGGAATTTAG-5′ | |

Mutants of STAT3 Decoy

| | | | |
|---|---|---|---|
| Mutant 1 | 5′-CAGTTCCCGTAAATC-3′ | 1 | +++ |
| (SEQ ID NOS:10, 11) | 3′-GTCAAGGGCATTTAG-5′ | | |
| Mutant 2 | 5′-CATTTCACGTAAATC-3′ | 1 | + |
| (SEQ ID NOS:12, 13) | 3′-GTAAAGTGCATTTAG-5′ | | |
| Mutant 3 | 5′-CATTTCCCTTAAATC-3′ | 1 | − |
| (SEQ ID NOS:14, 15) | 3′-GTAAAGGGAATTTAG-5′ | | |
| Mutant4 | 5′-CATTTCCCGTCAATC-3′ | 1 | ++ |
| (SEQ ID NOS:16, 17) | 3′-GTAAAGGGCAGTTAG-5′ | | |
| Mutant 5 | 5′-CAGTTCACGTAAATC-3′ | 2 | ++ |
| (SEQ ID NOS:18, 19) | 3′-GTCAAGTGCATTTAG-5′ | | |
| Mutant 6* | 5′-CAGTTCCCGTCAATC-3′ | 2 | + |
| (SEQ ID NOS:20, 21) | 3′-GTCAAGGGCAGTTAG-5′ | | |
| Mutant 7 | 5′-CATTTCACGTCAATC-3′ | 2 | + |
| (SEQ ID NOS:22, 23) | 3′-GTAAAGTGCAGTTAG-5′ | | |
| Mutant 8 | 5′-CATTTCCCTTCAATC-3′ | 2 | +++ |
| (SEQ ID NOS:24, 25) | 3′-GTAAAGGGAAGTTAG-5′ | | |
| Mutant 9 | 5′-CAGTTCACGTCAATC-3′ | 3 | +/− |
| (SEQ ID NOS:26, 27) | 3′-GTCAAGTGCAGTTAG-5′ | | |
| Mutant 10 | 5′-CAGTTCCCTTCAATC-3′ | 3 | +/− |
| (SEQ ID NOS:28, 29) | 3′-GTCAAGGGAAGTTAG-5′ | | |

*wild-type core sequence of SIF binding element of c-fos promoter: Wagner et al., EMBO J. 9(13):4477-4484 (1990).

Immunoblotting. Whole cell extracts were mixed with 2× sodium dodecyl sulfate (SDS) sample buffer (125 mmol/L Tris-HCL, pH 6.8; 4% SDS; 20% glycerol; 10% 2 mercaptoethanol) at 1:1 ratio and were heated for 5 min at 100° C. Proteins (50 μg/lane) were separated by 12.5% SDS-PAGE and transferred onto a nitrocellulose membrane (MSI; Westboro, Mass). Prestained molecular weight markers (GIBCO BRL; Gaithersburg, MD) were included in each gel. Membranes were blocked for 30 min in Tris-buffered saline (TBS: 10 mmol/L Tris-HCL, pH 7.5 and 150 mmol/L NaCl) with 0.5% Tween-20 (TBST) and 5% BSA. After blocking, membranes were incubated with a primary antibody; rabbit anti-human VEGF polyclonal antibody, rabbit anti-human Cyclin D1 polyclonal antibody, mouse anti-human Bcl-x_{L} monoclonal antibody, or rabbit anti-human PCNA polyclonal antibody (Santa Cruz Biotechnology, Santa Cruz, Calif.), in TBST and 1% BSA. After washing the membranes three times with TBST (5 min each), they were incubated with horseradish peroxidase-conjugated secondary antibody in TBST and 1% BSA for 30 min. Subsequently, membranes were washed three times with TBST and developed using the enhanced chemiluminescence (ECL) detection system (Amersham Life Sciences Inc.; Arlington Heights, Ill.). Quantification of Cyclin D1 and Bcl-x_{L} was performed by using a Molecular Dynamics Personal Densitometer SI and IMAGEQUANT software (Image Products International, Chantilly, Va.). Immunoblotting bands were quantified by densitometry and the Molecular Analyst software (AlphaDigidoc 1000, Alpho-Innotech) with normalization of each band to their corresponding loading control (Xi, S., et al. J Biol Chem, 278: 31574-31583, 2003).

Electrophoretic Mobility Shift Assay (EMSA). Whole cell extracts were prepared and EMSAs performed on 4% native polyacrylamide gels as described (Xi, S., et al. J Biol Chem, 278: 31574-31583, 2003 and Wong, P., etal. Mol Cell Biol, 14. 914-922, 1994). STAT3 activation was evaluated by using binding reactions with 20 μg of extracted protein, and radiolabeled high-affinity serum inducible element (hSIE) duplex oligonucleotide (Wagner et al., EMBO J. 9(13):4477-4484 (1990)). Quantification of the STAT3 signal was performed by scanning the SIF-A band using a Molecular Dynamics Personal Densitometer SI and IMAGEQUANT software. Normalization between blots was accomplished by running aliquots of U937 cell lysates (5 μg) that demonstrate activation of STAT3 on each gel. For supershift experiments, extracts were preincubated with STAT3 polyclonal antibody (C-20, Santa Cruz Biotechnology). Gel shift bands were quantified by densitometry and the Molecular Analyst software (AlphaDigidoc 1000, Alpha-Innotech) with normalization of each band to the positive control lysate run on that gel as described previously for STAT3 activation determinations (Grandis, J. R. et al. Proc Natl Acad Sci USA, 97: 4227-4232., 2000).

In vivo apoptosis determinations. Detection and quantitation of apoptosis was performed by the TUNEL reaction, using the In Situ Cell Death Detection Kit, POD (Roche Diagnostics GmbH, Roche Applied Science, Sandhofer Strasse 116, D-68305 Mannheim, Germany). Cryostat sections were fixed in 4% paraformaldehyde in PBS for 30 min. After washing with PBS for 30 min, the sections were incubated in permeabilization solution (0.1% Triton X-100, 0.1% sodium citrate) for 2 min on ice. The sections were washed twice with PBS before the samples were incubated for 60 min at 37° C. with TUNEL reaction mixture to label DNA strand breaks with Fluorescein-dUTP. After washing three times with PBS, the samples were incubated with Converter-POD for 30 min at 37° C., washed again with PBS, and finally exposed to 0.025% 3,3′-diaminobenzidine (DAB)/0.01% H₂O₂ in Tris-HCl buffer (pH 7.4) for 10 min. The number of apoptotic cells per high-power field (HPF) was counted. For each treatment, five samples were evaluated, and 5-10 fields of view were quantified on each section.

Statistics. In vivo comparisons of STAT3, cyclin D1 and Bcl-x_{L} expression levels were conducted with the Wilcoxon test for comparing two groups or the Kruskal-Wallis test for comparing three groups. All tests were exact and two-tailed. Studies of the joint effect of STAT3 decoy and cisplatin upon in vitro apoptosis were evaluated with a two way factorial design. Additive and synergistic effects of the STAT3 decoy and cisplatin were tested with a permutation test. In vivo tumor xenograft experiments were analyzed with mixed linear models that assumed animals were random effects having an unstructured within-animal covariance matrix. Data were log transformed and examined for the interaction between treatment group and day of observation beginning with day 10 after tumor inoculation, differences between treatment groups were estimated and tested with the pooled estimated standard error. Multiple comparisons were controlled by simulating observations from a multivariate t distribution with the same covariance matrix as the observed data and adjusting p values accordingly.

### Example 1

### STAT3 Treatment in Vivo

In vivo tumor xenograft studies. Female athymic nude mice nu/nu (4-6 weeks old; 20±2 g; Harlan-Sprague-Dawley) were implanted with 1×10⁶ 1483 cells into the right and left flank with a 26-gauge needle/1 ml tuberculin syringe, resulting in two tumors per mouse. Approximately 10 days later, when the tumor nodules were established (≈2×2 mm in diameter), mice were randomly assigned to treatment groups (STAT3 decoy, mutant control decoy). There were 10 mice in each treatment group. Twenty five micrograms of decoy (left flank) or mutant control decoy (right flank) oligonucleotide, suspended in water to a volume of 50 μl was delivered daily to the mice by intratumoral injection for 25 days. Mice were sacrificed after 25 days from the initial injection and tumors were harvested for analysis.

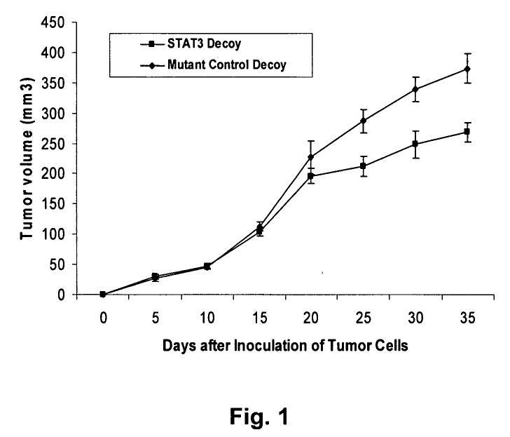

After approximately 10 treatments, the STAT3 decoy treated tumors were growth inhibited compared with the control treated tumors (p=0.002), an effect that persisted throughout treatment (FIG. 1). Tumors were harvested at the conclusion of each experiment (25 treatments) and stained for apoptotic cells by TUNEL. Treatment with STAT3 decoy resulted in 3.25-fold increase in apoptosis compared with mutant control decoy therapy (p=0.00038) (FIG. 2). These results demonstrate that the STAT3 decoy inhibits SCCHN tumor growth in vivo, at least in part, by inducing apoptosis.

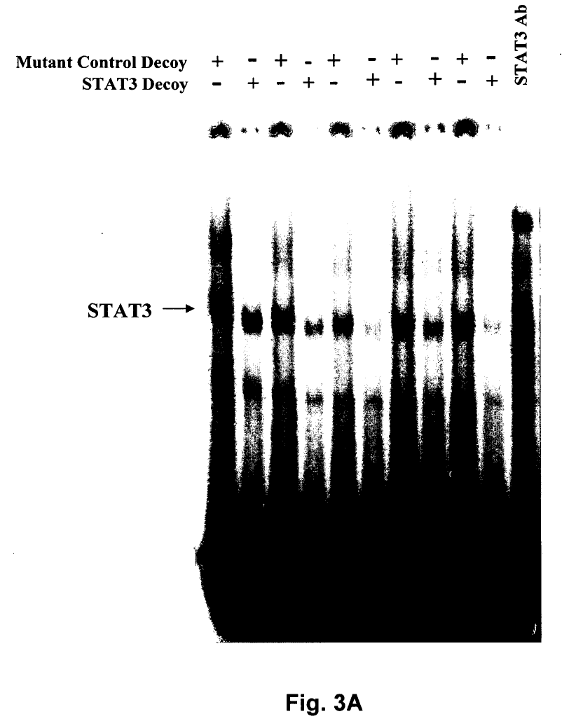

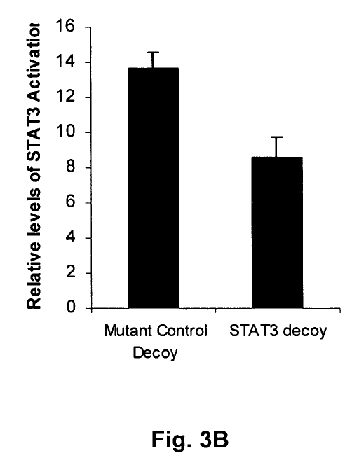

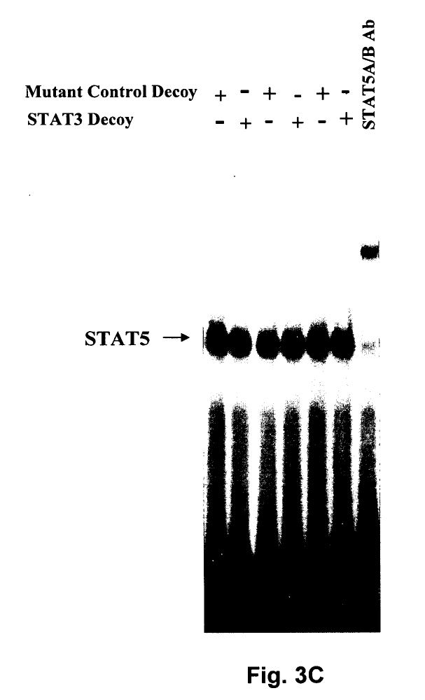

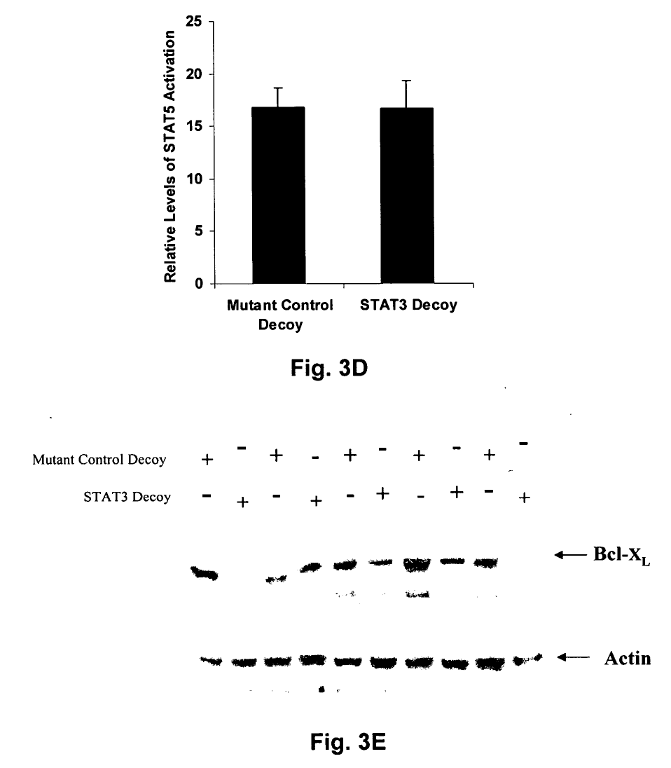

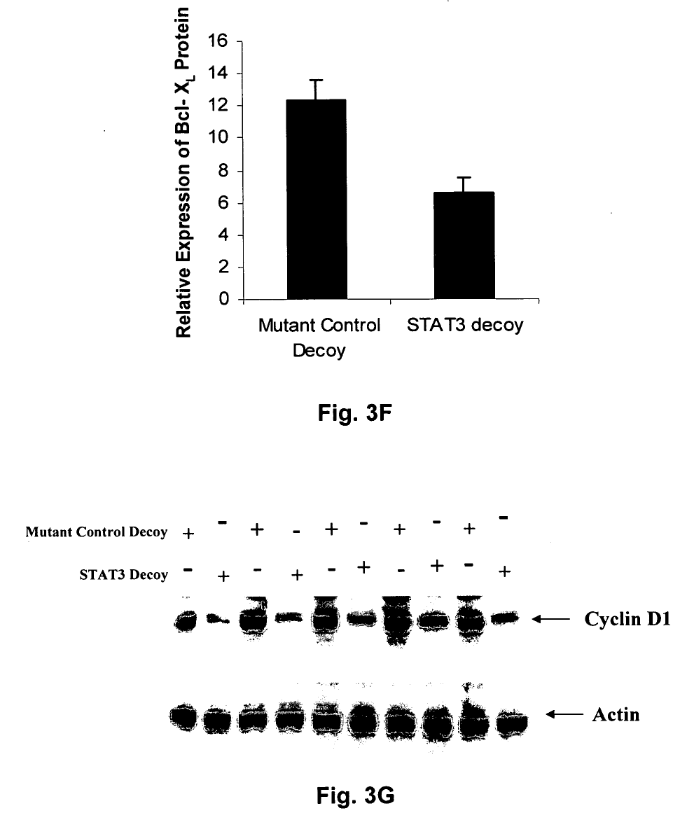

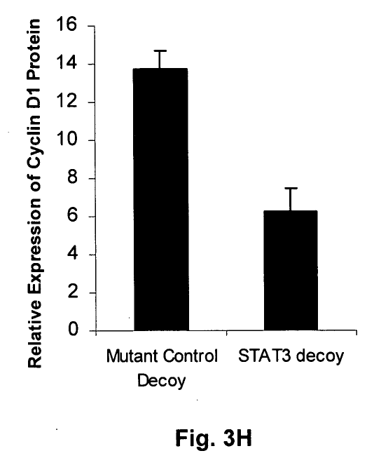

Mechanistic studies to evaluate the STAT3 decoy in vitro demonstrated that it worked by specifically inhibiting STAT3 activation and not by decreasing STAT3 expression or STAT3 tyrosine phosphorylation. Blockade of STAT3 should abrogate gene expression of targets that control cell survival and proliferation. To determine the effects of the STAT3 decoy in vivo, xenografts were harvested 4 hrs after the last treatment and processed for EMSA. As shown in FIGS. 3A-3H, treatment with the STAT3 decoy decreased STAT3 activation levels compared with treatment using the mutant control decoy (p=0.02). To demonstrate the specificity of the STAT3 decoy, the tumors were also analyzed for STAT5 activation and no abrogation was detected (FIGS. 3C and 3D). In addition, immunoblotting for Cyclin D1 and Bcl-x_{L} was performed to determine the effects of the STAT3 decoy on STAT3 target gene expression in vivo (FIGS. 3E-3H). An exact 2-tailed Wilcoxon test demonstrated a significant effect of the STAT3 decoy on Bcl-x_{L} (p=0.0002) and Cyclin D1 (p=0.0022) expression levels in the SCCHN xenografts treated with the STAT3 decoy compared to those tumors treated with the mutant control decoy.

### Example 2

### Decoy Plus Cisplatin in Vitro.

SCCHN cells (1483) were treated with mutant control decoy (25 μM), or STAT3 decoy alone (25 μM), or cisplatin alone (20 μM), or STAT3 decoy (25 μM) plus cisplatin (20 μM). Mutant control decoy or STAT3 decoy was always applied for a total of 6 days, and cisplatin was added for the last 24 hr before harvesting. The effects of the STAT3 decoy plus cisplatin on apoptosis was assessed by an Annexin 5-Cy3 apoptosis assay (BioVision Research Products, 2455-D Old Middlefield Way, Mountain View, Calif. 94043 USA) and Bcl-x_{L} and Cyclin D1 were examined using immunoblotting as described below.

Following treatment with STAT3 decoy plus cisplatin, SCCHN cells were detached by trypsinization, counted and pelleted (1000 r.p.m. for 5 min). Cell pellets were washed once with PBS (pH 7.4) and resuspended in 100 μl Annexin V binding buffer (10 mM HEPES, pH 7.4, 140 mM NaCl, 2.5 mM CaCl₂). 5×10⁵ cells were transferred to a 12×75 mm tube and 5 μl of Annexin V-Cy3 (BioVision Research Products, 2455-D Old Middlefield Way, Mountain View, Calif. 94043, USA) was added per tube and allowed to incubate at room temperature for 15 min in the dark. Then the stained cell suspension was dropped on the slides and covered with cover slips. The membranes of apoptotic cells are stained a bright orange color when analyzed with a fluorescence microscope. The ratio (percentage) of apoptotic to total cells (apoptotic plus non-apoptotic cells) was calculated for each high power field. For each treatment, 5-10 high power fields of view were quantitated on each section.

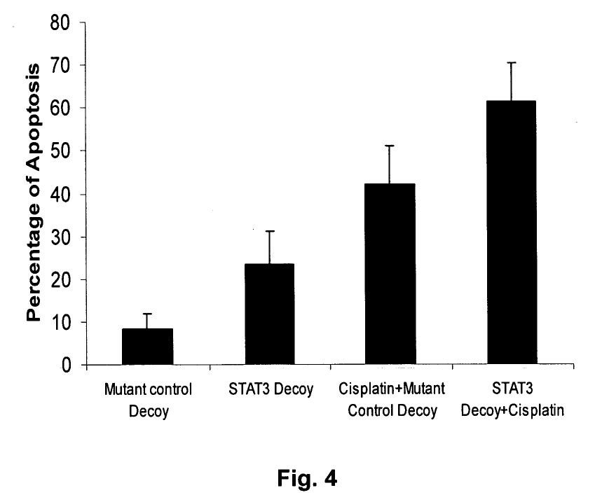

The effects of the STAT3 decoy plus cisplatin on apoptosis was examined by fluorescence microscopy (40X). As shown in FIG. 4, apoptosis was increased in the SCCHN cells treated with cisplatin plus the STAT3 decoy compared with the decoy alone (p=0.00015).

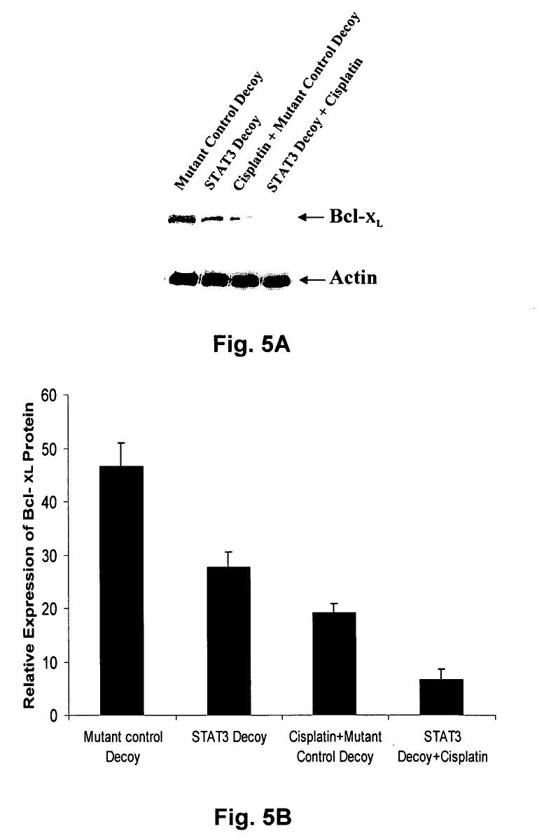

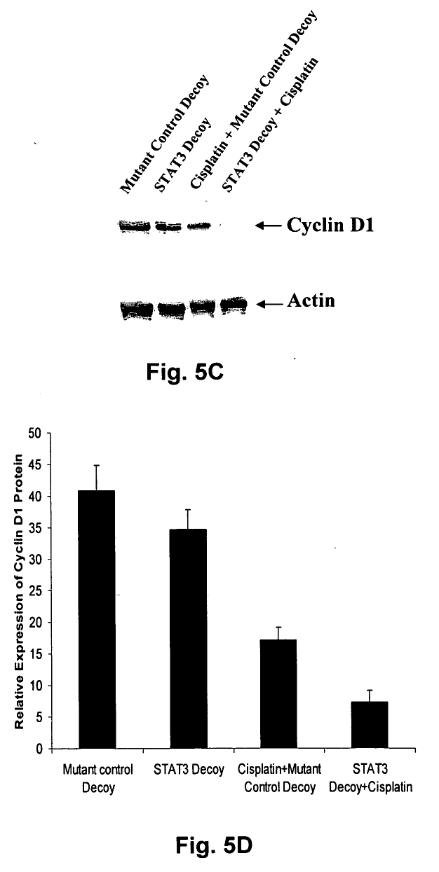

As shown above, treatment with the STAT3 decoy abrogates STAT3 target gene expression in vivo. To determine the effects of cisplatin in combination with the STAT3 decoy on STAT3 target gene expression, SCCHN cells were treated with the STAT3 decoy (or mutant control decoy), with or without cisplatin and harvested for immunoblotting (FIGS. 5A-5D). The data were analyzed using parametric analysis of variance of a two-way factorial design. In all three sets of experiments, the STAT3 decoy increased apoptosis and decreased Cyclin D1 and Bcl-x_{L} expression compared with the mutant control decoy (p<0.0001). Furthermore, the combination of the STAT3 decoy and cisplatin demonstrated an additive effect in all studies.

### Example 3

### STAT3 Decoy Plus Cisplatin in Vivo.

In an effort to maximize the therapeutic effects of the STAT3 decoy in vivo, the decoy was combined with cisplatin in a xenograft model of SCCHN. Mice were randomly assigned to treatment groups (STAT3 decoy, mutant control decoy, ciplatin alone, cisplatin plus STAT3 decoy, cisplatin plus mutant control decoy). There were 6-8 mice in each treatment group. Cisplatin (5 mg/kg) was injected intraperitoneally, and intratumoral injection of decoy (25 μg in water in a volume of 50 μl), was delivered as described in Example 1.

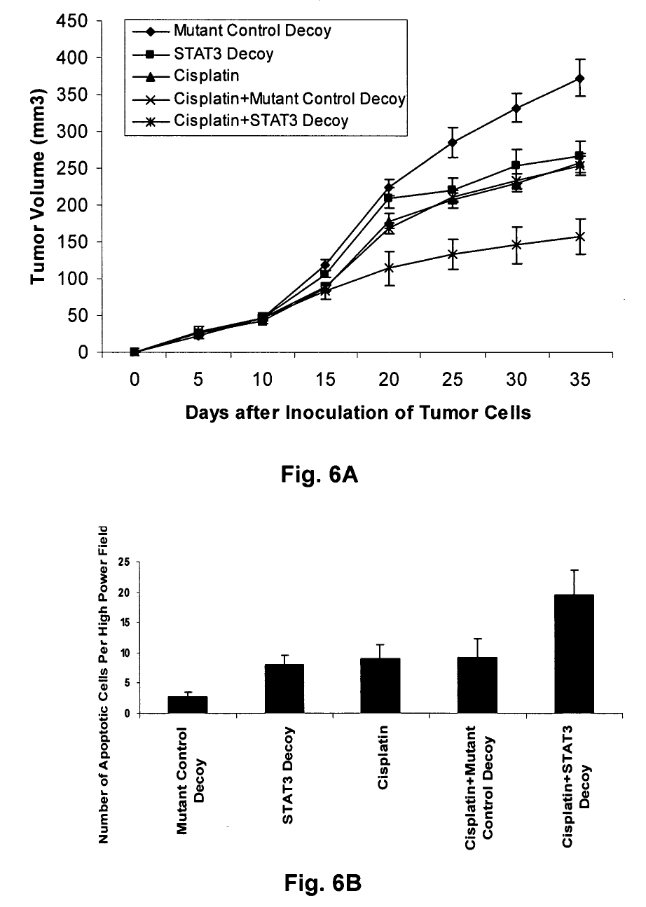

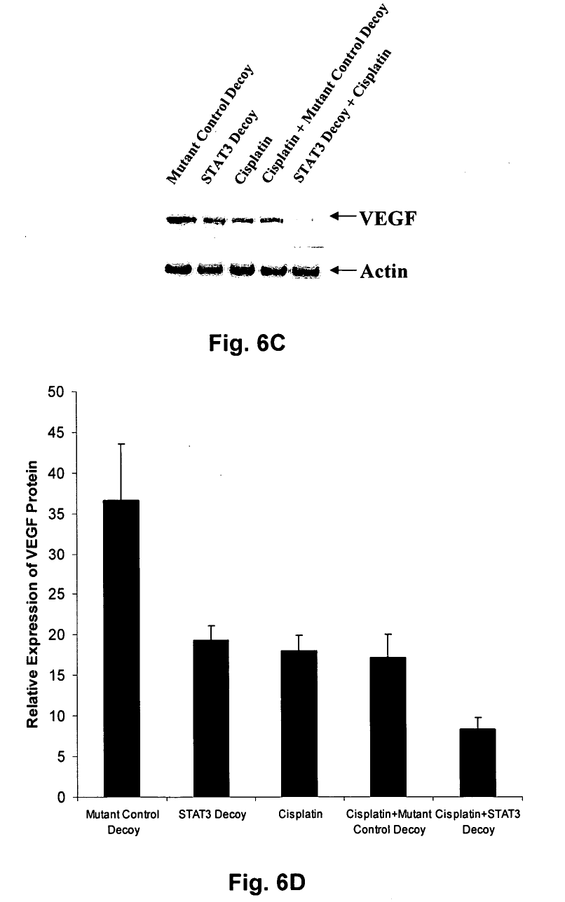

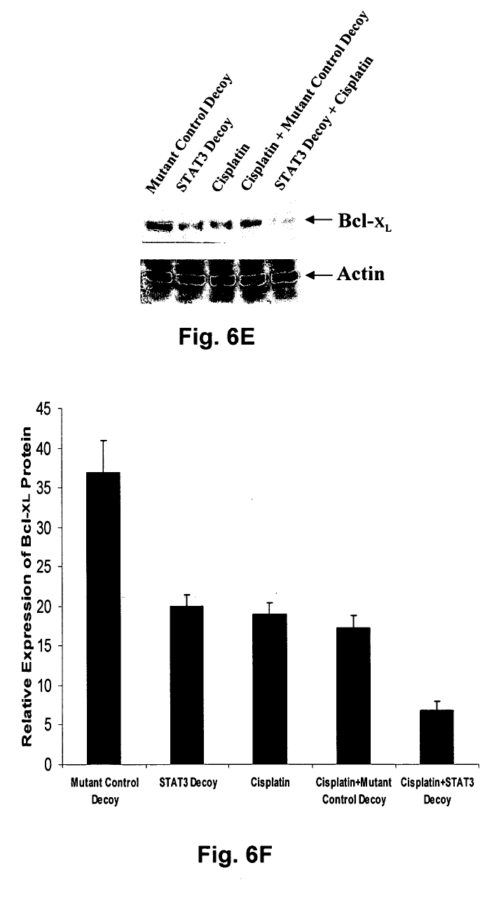

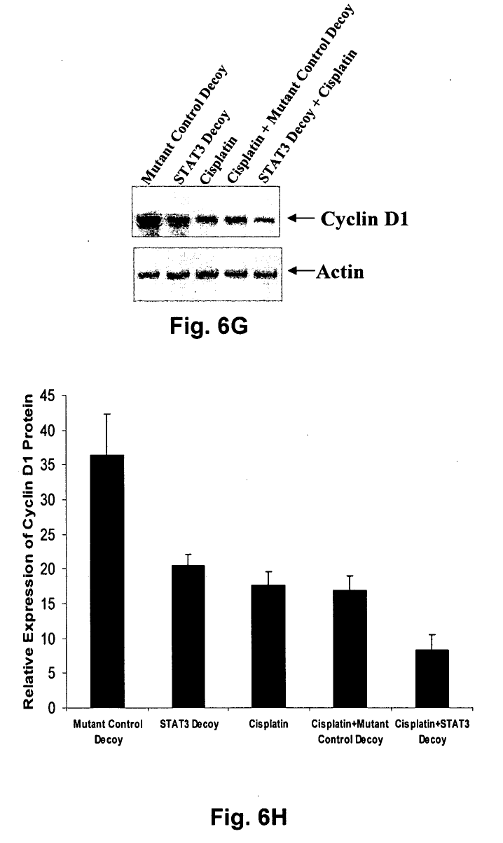

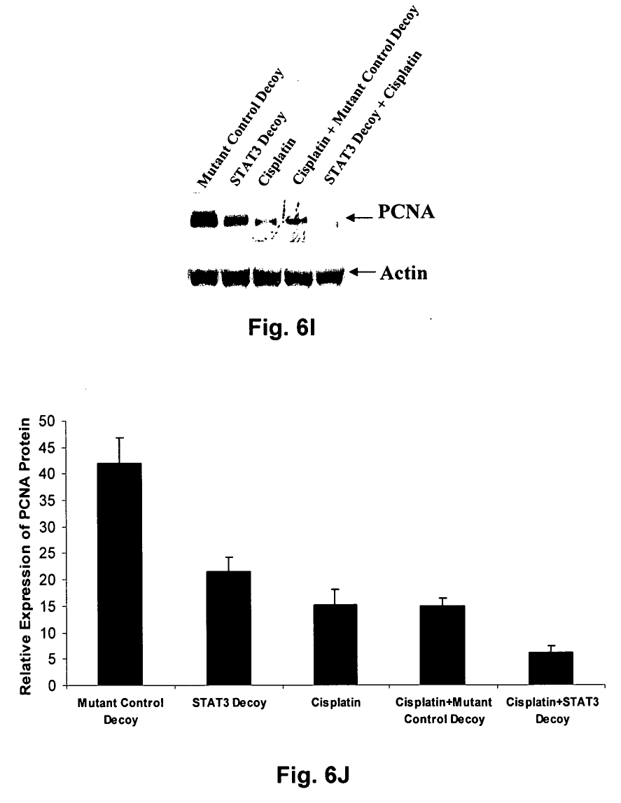

Ten days after initiating therapy in established tumors, the group receiving STAT3 decoy combined with cisplatin were growth inhibited compared with cisplatin combined with mutant control decoy or cisplatin alone, an effect that persisted throughout treatment (FIG. 6A) (p=0.02). Tumors were harvested at the conclusion of each experiment (25 treatments) and stained for apoptotic cells by TUNEL (FIG. 6B). These results demonstrate that the STAT3 decoy combined with cisplatin had inhibition on SCCHN tumor growth in vivo, at least in part by inducing apoptosis (7.4-fold induction, p=0.002). VEGF has also been reported as a STAT3 target gene, and would be expected to play a role in STAT3-mediated effects in vivo. The consequences of the combined decoy/cisplatin therapy on VEGF (FIGS. 6C and 6D), Bcl-x_{L} (FIGS. 6E and 6F), Cyclin D1 (FIGS. 6G and 6H) and PCNA (FIGS. 61 and 6J) expression was examined in vivo. An exact 2-tailed Wilcoxon test demonstrated a significant effect of the STAT3 decoy combined with cisplatin on VEGF (p=0.0004), Bcl-x_{L} (p=0.0001), Cyclin D1 (p=0.00038), and PCNA (p=0.00054) expression levels in the SCCHN xenografts treated with the STAT3 decoy combined with cisplatin compared to those tumors treated with the STAT3 decoy combined with mutant control decoy or cisplatin alone.

### Example 4

### Dose Response Curve for Gossypol

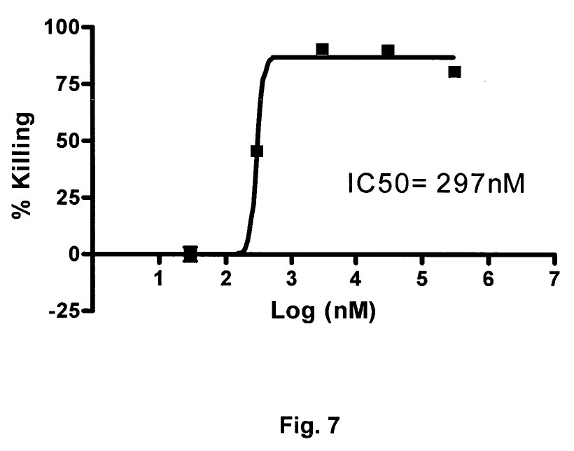

Dose response experiments have been completed for gossypol using UM-22B, PCI-5B, and 1483 cell lines and determined an average IC₅₀ value of 297 nM for PCI-15B cells at day 3 after treatment, as determined by both MTT assays (3-(4,5-dimethylthiazol-2-yl)-2,5-diphenyltetrazolium bromide assay) and cell counting experiments (see, for example, FIG. 7). In FIG. 7, PCI-15B cells were treated with 0, 0.003, 0.03, 3, and 30 μM gossypol. After 3 days, MTT assays and cell counting experiments were performed. The IC₅₀ value was then calculated as 297 nM. Bars indicate standard error. Similar results were seen for UM-22B and 1483 cells (data not shown). The STAT3 decoy will be combined with gossypol to determine the antitumor efficacy of the combination treatment.

### Example 5

### Action of STAT3 Decoy on p53, GAS and Egr-1 Response/promoter Elements and Interaction with STAT1

The STAT3 decoy may exert its anti-tumor activity via mechanisms in addition to direct inhibition of STAT3 DNA binding and target gene expression. It was hypothesized that the STAT3 decoy may interfere with the function of other protein(s) that interact with STAT3 protein, thus resulting in STAT3-specific anti-tumor activity. To this end, experiments were conducted to determine the effect of the STAT3 decoy on p53 and GAS (Interferon-Gamma Activated Sequence) promoter activity and whether the STAT3 decoy is able to bind STAT 1, a protein known to interact with STAT3.

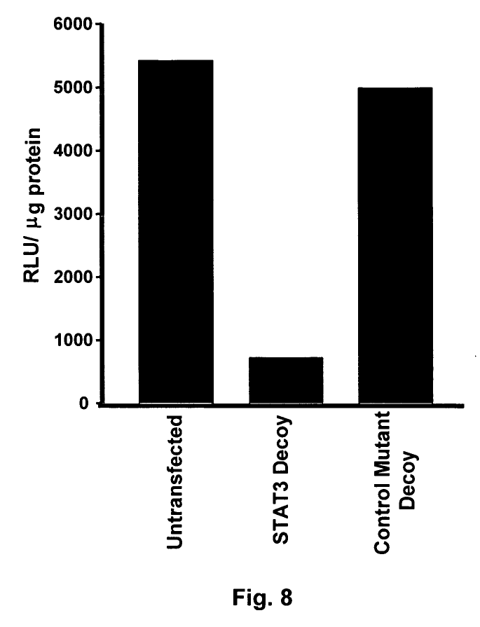

FIG. 8 depicts the inhibition of reporter gene expression from a p53-responsive element in SCCHN cells. An SCCHN cell line, UM-22B, was used to generate a stable cell line expressing a p53-Luciferase (p53-Luc) reporter gene (The p53-Luc plasmid carrying 14 repeats of p53 transcription recognition sequence: 5′-TGCCTGGACTTGCCTGG-3 (SEQ ID NO: 30)), was obtained from Stratagene, Catalog number: 219085-51). The cells were transfected with 2 μg of the STAT3 decoy or the control mutant decoy. At 24 hrs after transfection, luciferase (Luc) activity was measured and normalized to the total amount of protein according to standard methods.

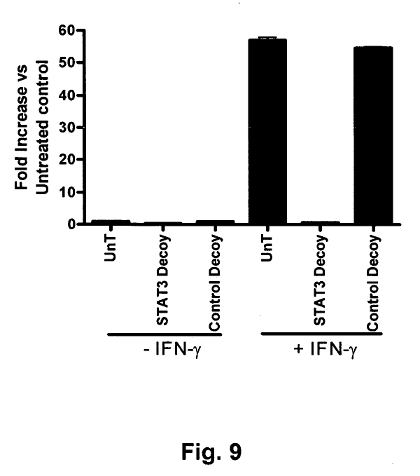

FIG. 9 depicts the inhibition of reporter gene expression from GAS-responsive element in SCCHN cells. An SCCHN cell line, UM-22B, was used to generate a stable cell line expressing a pGAS-Luc reporter gene (pGAS-Luc plasmid carrying 4 repeats of GAS sequence: 5′-AGTTTCATATTACTCTAAATC-3′ (SEQ ID NO: 31), was obtained from Stratagene, Catalog number: 219091-51). The cells were transfected with 2 μg of the STAT3 decoy or the control mutant decoy. At 24 hrs after transfection, luciferase activity was measured and normalized to the total amount of protein.

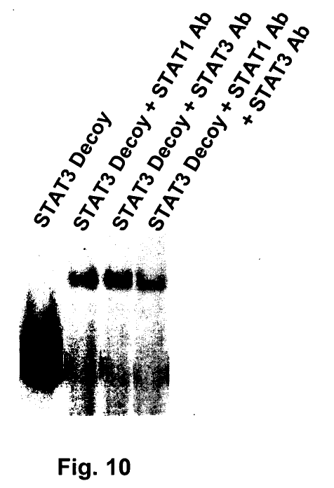

FIG. 10 depicts binding of STAT 1 to the STAT3 decoy in an electrophoretic mobility shift assay (EMSA). Nuclear protein extracts of PCI-15B cells, prepared according to standard methods, were used for the EMSA. The specificity of STAT1 or STAT3 binding to the STAT3 decoy was determined by supershifting with STAT1- or STAT3-specific antibodies, or both. The experiment has been repeated more than 3 times.

As shown in FIGS. 8, 9 and 10, the STAT3 decoy, but not the control mutant decoy, can specifically interfere with p53 and STAT1 reporter activity. In addition, the STAT3 decoy, but not the control mutant decoy, is able to bind to STAT1 (a known STAT3 interacting protein) and inhibit reporter gene expression from a GAS-responsive element upon IFN-γ stimulation in SCCHN cells (FIG. 9 and 10).

STAT3 decoy-specific transcriptional repression of Egr-1(Early Growth Response -1 enhancer, pEGR-1-Luc, Stratagene Cat. No. 240130, containing three repeats of the Egr-1 transcription recognition sequence: 5′-GGGTGGGGN-3′ (SEQ ID NO: 31) was also observed. SCCHN cells were co-transfected with 2 μg pEgr-1-Luc plasmid and 2 μg of the STAT3 decoy or the control mutant decoy. At 24 hrs after transfection, luciferase activity was measured and normalized to the total amount of protein according to standard methods.

Having now fully described this invention, it will be understood to those of ordinary skill in the art that the same can be performed within a wide and equivalent range of conditions, formulations and other parameters without affecting the scope of the invention or any embodiment thereof.

We claim:

1. A composition comprising an amount of a STAT3 decoy effective to reduce growth of a cancer in which STAT3 is activated in a patient and a pharmaceutically acceptable carrier.

2. The composition of claim 1, further comprising an anticancer agent.

3. The composition of claim 2, wherein the anticancer agent is selected from the group consisting of: tyrosine kinase inhibitors; antibodies or fragments thereof; angiogenesis inhibitors; immune modulators; non-tyrosine kinase inhibitors; serine/threonine kinase inhibitors; farnesyl or geranyl transferase inhibitors, such as FTI-277 and GGTI-298; and G-protein-coupled receptor inhibitors.

4. The composition of claim 1, further comprising an anticancer agent selected from the group consisting of aldesleukin; alemtuzumab; alitretinoin; allopurinol; altretarnine; amifostine; An-238; anastrozole; arsenic trioxide; asparaginase; BCG Live; bevacizumab; bexarotene; bleomycin; busulfan; calusterone; capecitabine; capecitabine; carboplatin; carmustine; celecoxib; cetuximab; chlorambucil; cisplatin; cladribine; cyclophosphamide; cyclophosphamide; cytarabine; dactinomycin; darbepoetin alfa; daunorubicin; daunorubicin, daunomycin; denileukin diftitox; dexrazoxane; docetaxel; doxorubicin; dromostanolone propionate; Elliott's B Solution; endostatin; epirubicin; epoetin alfa; estramustine; etoposide phosphate; etoposide, VP-16; exemestane; filgrastim; floxuridine; fludarabine; fluorouracil; FTI-277; fulvestrant; gefitinib; gemcitabine; gemcitabine; gemtuzumab ozogamicin; GGTI-298; goserelin acetate; hydroxyurea; ibritumomab; idarubicin; idarubicin; ifosfamide; imatinib mesylate; interferon alfa-2a; interferon alfa-2b; IL-2; IL-12; irinotecan; letrozole; leucovorin; levamisole; lomustine; meclorethamine; nitrogen mustard; megestrol acetate; melphalan, L-PAM; mercaptopurine, 6-MP; mesna; methotrexate; methoxsalen; mitomycin C; mitotane; mitoxantrone; nandrolone phenpropionate; nofetumomab; oprelvekin; oxaliplatin; paclitaxel; pamidronate; pegademase; pegaspargase; pegfilgrastim; pentostatin; pentostatin; pipobroman; plicamycin; mithramycin; porfimer sodium; PP2; procarbazine; quinacrine; rasburicase; RC3095; rituximab; sargramostim; streptozocin; talc; tamoxifen; temozolomide; teniposide, VM-26; testolactone; thioguanine, 6-TG; thiotepa; topotecan; toremifene; tositumomab; trastuzumab; tretinoin, ATRA; UO126; uracil mustard; valrubicin; vinblastine; vincristine; vinorelbine; wortmanin and zoledronate.

5. The composition of claim 1, formulated as a parenteral dosage form.

6. The composition of claim 5, formulated as one of an intravenous and an intratumor dosage form.

7. The composition of claim 1, further comprising gossypol.

8. The composition of claim 1, further comprising cisplatin.

9. The composition of claim 8, comprising about 1 mg/mL cisplatin.

10. The composition of claim 1, wherein the cancer is a squamous cell carcinoma.

11. The composition of claim 1, wherein the STAT3 decoy is a double-stranded DNA or an analog thereof.

12. The composition of claim 11, wherein the STAT3 decoy is a DNA analog.

13. The composition of claim 12, wherein the DNA analog is a phosphorothioate nucleic acid analog.

14. The composition of claim 1, wherein the STAT3 decoy is a double-stranded DNA or an analog thereof comprising the STAT3 target sequence:
(SEQ ID NO:1)
5′-(N₆)ₙ-CAN₁TTCN₂CN₃TN₄AN₅TC-(N₇)ₘ-3′,
wherein N₁, N₂, N₃, N₄ and N₅ are A, T, G or C, and one, two, three or all of the following conditions are met: N₁, is T; N₂ is C; N₃ is G, N₄ is A and N₅ is A, and N₆ and N₇ are A, T, G or C and n and m are independently 0-50.

15. The composition of claim 14, wherein N₂ is a pyrimidine.

16. The composition of claim 14, wherein at least two of the following are met: N₁, is T; N₂ is C; N₃ is G, N₄ is A and N₅ is A.

17. The composition of claim 14, wherein at least three of the following are met: N₁, is T; N₂ is C; N₃ is G, N₄ is A and N₅ is A.

18. The composition of claim 14, wherein N₃ is G.

19. The composition of claim 1, comprising a double-stranded DNA acid or an analog thereof comprising a derivative of the STAT3 target sequence:
(SEQ ID NO:2)
5′-(N₆)ₙ-CATTTCCCGTAAATC-(N₇)ₘ-3′,
in which N₆ and N₇ are A, T, G or C and n and m are independently 0-50, containing a single nucleotide insertion, deletion or substitution within the sequence 5′-CATTTCCCGTAAATC-3′ (SEQ ID NO: 2).

20. The composition of claim 1, wherein the STAT3 decoy comprises a nucleic acid comprising one or more STAT3 sequences.

21. The composition of claim 20, wherein the STAT3 decoy comprises two or more STAT3 target sequences.

22. A composition comprising an amount of a STAT3 decoy effective to interfere with STAT3 binding to a STAT3 response element in a cancer cell of a patient and a pharmaceutically acceptable carrier.

23. The composition of claim 22, further comprising an anticancer agent.

24. The composition of claim 23, wherein the anticancer agent is selected from the group consisting of: tyrosine kinase inhibitors; antibodies or fragments thereof; angiogenesis inhibitors; immune modulators; non-tyrosine kinase inhibitors; serine/threonine kinase inhibitors; famesyl or geranyl transferase inhibitors, such as FTI-277 and GGTI-298; and G-protein-coupled receptor inhibitors.

25. The composition of claim 22, further comprising an anticancer agent selected from the group consisting of aldesleukin; alemtuzumab; alitretinoin; allopurinol; altretamine; amifostine; An-238; anastrozole; arsenic trioxide; asparaginase; BCG Live; bevacizumab; bexarotene; bleomycin; busulfan; calusterone; capecitabine; capecitabine; carboplatin; carmustine; celecoxib; cetuximab; chlorambucil; cisplatin; cladribine; cyclophosphamide; cyclophosphamide; cytarabine; dactinomycin; darbepoetin alfa; daunorubicin; daunorubicin, daunomycin; denileukin diftitox; dexrazoxane; docetaxel; doxorubicin; dromostanolone propionate; Elliott's B Solution; endostatin; epirubicin; epoetin alfa; estramustine; etoposide phosphate; etoposide, VP-16; exemestane; filgrastim; floxuridine; fludarabine; fluorouracil; FTI-277; fulvestrant; gefitinib; gemcitabine; gemcitabine; gemtuzumab ozogamicin; GGTI-298; goserelin acetate; hydroxyurea; ibritumomab; idarubicin; idarubicin; ifosfamide; imatinib mesylate; interferon alfa-2a; interferon alfa-2b; IL-2; IL-12; irinotecan; letrozole; leucovorin; levamisole; lomustine; meclorethamine; nitrogen mustard; megestrol acetate; melphalan, L-PAM; mercaptopurine, 6-MP; mesna; methotrexate; methoxsalen; mitomycin C; mitotane; mitoxantrone; nandrolone phenpropionate; nofetumomab; oprelvekin; oxaliplatin; paclitaxel; pamidronate; pegademase; pegaspargase; pegfilgrastim; pentostatin; pentostatin; pipobroman; plicamycin; mithramycin; porfimer sodium; PP2; procarbazine; quinacrine; rasburicase; RC3095; rituximab; sargramostim; streptozocin; talc; tamoxifen; temozolomide; teniposide, VM-26; testolactone; thioguanine, 6-TG; thiotepa; topotecan; toremifene; tositumomab; trastuzumab; tretinoin, ATRA; UO126; uracil mustard; valrubicin; vinblastine; vincristine; vinorelbine; wortmanin and zoledronate.

26. The composition of claim 22 formulated as a parenteral dosage form.

27. The composition of claim 26, formulated as one of an intravenous and an intratumor dosage form.

28. The composition of claim 22, further comprising gossypol.

29. The composition of claim 22, further comprising cisplatin.

30. The composition of claim 29, comprising about 1 mg/mL cisplatin.

31. The composition of claim 22, wherein the cancer cell is a cell of a squamous cell carcinoma.

32. The composition of claim 22, wherein the STAT3 decoy is a double-stranded DNA or an analog thereof.

33. The composition of claim 32, wherein the STAT3 decoy is a DNA analog.

34. The composition of claim 33, wherein the DNA analog is a phosphorothioate nucleic acid analog.

35. The composition of claim 22, wherein the STAT3 decoy is a double-stranded DNA or an analog thereof comprising the STAT3 target sequence:
(SEQ ID NO:1)
5′-(N₆)ₙ-CAN₁TTCN₂CN₃TN₄AN₅TC-(N₇)ₘ-3′,
wherein N₁, N₂, N₃, N₄ and N₅ are A, T, G or C, and one, two, three or all of the following conditions are met: N₁, is T; N₂ is C; N₃ is G, N₄ is A and N₅ is A, and N₆ and N₇ are A, T, G or C and n and m are independently 0-50.

36. The composition of claim 35, wherein N₂ is a pyrimidine.

37. The composition of claim 35, wherein at least two of the following are met: N₁, is T; N₂ is C; N₃ is G, N₄ is A and N₅ is A.

38. The composition of claim 35, wherein at least three of the following are met: N₁, is T; N₂ is C; N₃ is G,N₄ is A and N₅ is A.

39. The composition of claim 35, wherein N₃ is G.

40. The composition claim 22, comprising a double-stranded DNA or an analog thereof comprising a derivative of the STAT3 target sequence:
(SEQ ID NO:2)
5′-(N₆)ₙ-CATTTCCCGTAAATC-(N₇)ₘ-3′,
or a sequence complementary thereto, in which N₆ and N₇ are A, T, G or C and n and m are independently 0-50, containing a single nucleotide insertion, deletion or substitution within the sequence 5′-CATTTCCCGTAAATC-3′ (SEQ ID NO: 2).

41. The composition of claim 22, wherein the STAT3 decoy comprises a nucleic acid comprising one or more STAT3 target sequences.

42. The composition of claim 41, wherein the STAT3 decoy comprises two or more STAT3 target sequences.

43. A composition comprising an amount of a STAT3 decoy effective to induce apoptosis in a cancer cell of a patient in which STAT3 is activated and a pharmaceutically acceptable carrier.

44. The composition of claim 43, further comprising an anticancer agent.

45. The composition of claim 44, wherein the anticancer agent is selected from the group consisting of: tyrosine kinase inhibitors; antibodies or fragments thereof; angiogenesis inhibitors; immune modulators; non-tyrosine kinase inhibitors; serine/threonine kinase inhibitors; farnesyl or geranyl transferase inhibitors, such as FTI-277 and GGTI-298; and G-protein-coupled receptor inhibitors.

46. The composition of claim 43, further comprising an anticancer agent selected from the group consisting of aldesleukin; alemtuzumab; alitretinoin; allopurinol; altretamine; amifostine; An-238; anastrozole; arsenic trioxide; asparaginase; BCG Live; bevacizumab; bexarotene; bleomycin; busulfan; calusterone; capecitabine; capecitabine; carboplatin; carmustine; celecoxib; cetuximab; chlorambucil; cisplatin; cladribine; cyclophosphamide; cyclophosphamide; cytarabine; dactinomycin; darbepoetin alfa; daunorubicin; daunorubicin, daunomycin; denileukin diftitox; dexrazoxane; docetaxel; doxorubicin; dromostanolone propionate; Elliott's B Solution; endostatin; epirubicin; epoetin alfa; estramustine; etoposide phosphate; etoposide, VP-16; exemestane; filgrastim; floxuridine; fludarabine; fluorouracil; FTI-277; fulvestrant; gefitinib; gemcitabine; gemcitabine; gemtuzumab ozogamicin; GGTI-298; goserelin acetate; hydroxyurea; ibritumomab; idarubicin; idarubicin; ifosfamide; imatinib mesylate; interferon alfa-2a; interferon alfa-2b; IL-2; IL-12; irinotecan; letrozole; leucovorin; levamisole; lomustine; meclorethamine; nitrogen mustard; megestrol acetate; melphalan, L-PAM; mercaptopurine, 6-MP; mesna; methotrexate; methoxsalen; mitomycin C; mitotane; mitoxantrone; nandrolone phenpropionate; nofetumomab; oprelvekin; oxaliplatin; paclitaxel; pamidronate; pegademase; pegaspargase; pegfilgrastim; pentostatin; pentostatin; pipobroman; plicamycin; mithramycin; porfimer sodium; PP2; procarbazine; quinacrine; rasburicase; RC3095; rituximab; sargramostim; streptozocin; talc; tamoxifen; temozolomide; teniposide, VM-26; testolactone; thioguanine, 6-TG; thiotepa; topotecan; toremifene; tositumomab; trastuzumab; tretinoin, ATRA; UO126; uracil mustard; valrubicin; vinblastine; vincristine; vinorelbine; wortmanin and zoledronate.

47. The composition of claim 43, formulated as a parenteral dosage form.

48. The composition of claim 46, formulated as one of an intravenous and an intratumor dosage form.

49. The composition of claim 43, further comprising gossypol.

50. The composition of claim 43, further comprising cisplatin.

51. The composition of claim 50, comprising about 1 mg/mL cisplatin.

52. The composition of claim 43, wherein the cell is a cell of a squamous cell carcinoma.

53. The composition of claim 43, wherein the STAT3 decoy is a double-stranded DNA or an analog thereof.

54. The composition of claim 53, wherein the STAT3 decoy is an DNA analog.

55. The composition of claim 54, wherein the DNA analog is a phosphorothioate nucleic acid analog.

56. The composition of claim 43, wherein the STAT3 decoy is a double-stranded DNA or an analog thereof comprising the STAT3 target sequence:
(SEQ ID NO:1)
5′-(N₆)ₙ-CAN₁TTCN₂CN₃TN₄AN₅TC-(N₇)ₘ-3′,
wherein N₁, N₂, N₃, N₄ and N₅ are A, T, G or C, and one, two, three or all of the following conditions are met: N₁, is T; N₂ is C; N₃ is G, N₄ is A and N₅ is A, and N₆ and N₇ are A, T, G or C and n and m are independently 0-50.

57. The composition of claim 56, wherein N₂ is a pyrimidine.

58. The composition of claim 56, wherein at least two of the following are met: N₁, is T; N₂ is C; N₃ is G, N₄ is A and N₅ is A.

59. The composition of claim 56 wherein at least three of the following are met: N₁, is T; N₂ is C; N₃ is G, N₄ is A and N₅ is A.

60. The composition of claim 56, wherein N₃ is G.

61. The composition of claim 43, comprising a double-stranded DNA or an analog thereof comprising a derivative of the STAT3 target sequence:
(SEQ ID NO:2)
5′-(N₆)ₙ-CATTTCCCGTAAATC-(N₇)ₘ-3′,
or a sequence complementary thereto, in which N₆ and N₇ are A, T, G or C and n and m are independently 0-50, containing a single nucleotide insertion, deletion or substitution within the sequence 5′-CATTTCCCGTAAATC-3′ (SEQ ID NO: 2).

62. The composition of claim 43, wherein the STAT3 decoy comprises a nucleic acid comprising one or more STAT3 target sequences.

63. The composition of claim 62, wherein the STAT3 decoy comprises two or more STAT3 target sequences.

64. A method of reducing growth of a cancer in which STAT3 is activated in a patient, comprising administering to the patient an amount of the composition of claim 1 effective to reduce growth of a cancer in a patient, thereby reducing growth of the cancer in the patient.

65. The method of claim 64, wherein the cancer is a squamous cell carcinoma.

66. The method of claim 64, wherein the cancer is a squamous cell carcinoma of the head and neck.

67. The method of claim 64, comprising administering to the patient a second anticancer therapy.

68. The method of claim 67, wherein the second anticancer therapy is one or both of a radiation therapy and treating the patient with an anticancer agent.

69. The method of claim 68, wherein the second anticancer therapy is a radiation therapy.

70. The method of claim 68, wherein the second anticancer therapy comprises treating the patient with an anticancer agent.

71. The method of claim 70, wherein the anticancer agent is selected from the group consisting of aldesleukin; alemtuzumab; alitretinoin; allopurinol; altretamine; amifostine; An-238; anastrozole; arsenic trioxide; asparaginase; BCG Live; bevacizumab; bexarotene; bleomycin; busulfan; calusterone; capecitabine; capecitabine; carboplatin; carmustine; celecoxib; cetuximab; chlorambucil; cisplatin; cladribine; cyclophosphamide; cyclophosphamide; cytarabine; dactinomycin; darbepoetin alfa; daunorubicin; daunorubicin, daunomycin; denileukin diftitox; dexrazoxane; docetaxel; doxorubicin; dromostanolone propionate; Elliott's B Solution; endostatin; epirubicin; epoetin alfa; estramustine; etoposide phosphate; etoposide, VP-16; exemestane; filgrastim; floxuridine; fludarabine; fluorouracil; FTI-277; fulvestrant; gefitinib; gemcitabine; gemcitabine; gemtuzumab ozogamicin; GGTI-298; goserelin acetate; hydroxyurea; ibritumomab; idarubicin; idarubicin; ifosfamide; imatinib mesylate; interferon alfa-2a; interferon alfa-2b; IL-2; IL-12; irinotecan; letrozole; leucovorin; levamisole; lomustine; meclorethamine; nitrogen mustard; megestrol acetate; melphalan, L-PAM; mercaptopurine, 6-MP; mesna; methotrexate; methoxsalen; mitomycin C; mitotane; mitoxantrone; nandrolone phenpropionate; nofetumomab; oprelvekin; oxaliplatin; paclitaxel; pamidronate; pegademase; pegaspargase; pegfilgrastim; pentostatin; pentostatin; pipobroman; plicamycin; mithramycin; porfimer sodium; PP2; procarbazine; quinacrine; rasburicase; RC3095; rituximab; sargramostim; streptozocin; talc; tamoxifen; temozolomide; teniposide, VM-26; testolactone; thioguanine, 6-TG; thiotepa; topotecan; toremifene; tositumomab; trastuzumab; tretinoin, ATRA; UO126; uracil mustard; valrubicin; vinblastine; vincristine; vinorelbine; wortmanin and zoledronate.

72. The method of claim 70, wherein the anticancer agent is cisplatin.

73. The method of claim 70, wherein the anticancer agent is gossypol.

74. The method of claim 70, wherein the anticancer agent is selected from the group consisting of: tyrosine kinase inhibitors; antibodies or fragments thereof; angiogenesis inhibitors; immune modulators; non-tyrosine kinase inhibitors; serine/threonine kinase inhibitors; farnesyl or geranyl transferase inhibitors, such as FTI-277 and GGTI-298; and G-protein-coupled receptor inhibitors.

75. The method of claim 64, wherein the cancer is selected from the group consisting of multiple myeloma; HTLV-1 dependent leukemia; acute myelogenous leukemia; large granular lymphocyte leukemia; lymphoma; EBV-related Burkitt's lymphoma; mycosis fungoides; cutaneous T-cell lymphoma; non-Hodgkins lymphoma; anaplastic large-cell lymphoma; breast cancer; melanoma; ovarian cancer; lung cancer; pancreatic cancer and prostate cancer.

76. A method of interfering with STAT3 binding to a STAT3 response element in a cancer cell of a patient in which STAT3 is activated, comprising administering to the patient an amount of the composition of claim 20 effective to interfere with STAT3 binding to a STAT3 response element in the cancer cell, thereby interfering with STAT3 binding to a STAT3 response element in the cancer cell.

77. The method of claim 76, wherein the cancer cell is a cell of a squamous cell carcinoma.

78. The method of claim 76, wherein the cancer cell is a cell of a squamous cell carcinoma of the head and neck.

79. The method of claim 76, comprising administering to the patient a second anticancer therapy.

80. The method of claim 79, wherein the second anticancer therapy is one or both of a radiation therapy and treating the patient with an anticancer agent.

81. The method of claim 80, wherein the second anticancer therapy is a radiation therapy.

82. The method of claim 80, wherein the second anticancer therapy comprises treating the patient with an anticancer agent.

83. The method of claim 82, wherein the anticancer agent is selected from the group consisting of aldesleukin; alemtuzumab; alitretinoin; allopurinol; altretamine; amifostine; An-238; anastrozole; arsenic trioxide; asparaginase; BCG Live; bevacizumab; bexarotene; bleomycin; busulfan; calusterone; capecitabine; capecitabine; carboplatin; carmustine; celecoxib; cetuximab; chlorambucil; cisplatin; cladribine; cyclophosphamide; cyclophosphamide; cytarabine; dactinomycin; darbepoetin alfa; daunorubicin; daunorubicin, daunomycin; denileukin diftitox; dexrazoxane; docetaxel; doxorubicin; dromostanolone propionate; Elliott's B Solution; endostatin; epirubicin; epoetin alfa; estramustine; etoposide phosphate; etoposide, VP-16; exemestane; filgrastim; floxuridine; fludarabine; fluorouracil; FTI-277; fulvestrant; gefitinib; gemcitabine; gemcitabine; gemtuzumab ozogamicin; GGTI-298; goserelin acetate; hydroxyurea; ibritumomab; idarubicin; idarubicin; ifosfamide; imatinib mesylate; interferon alfa-2a; interferon alfa-2b; IL-2; IL-12; irinotecan; letrozole; leucovorin; levamisole; lomustine; meclorethamine; nitrogen mustard; megestrol acetate; melphalan, L-PAM; mercaptopurine, 6-MP; mesna; methotrexate; methoxsalen; mitomycin C; mitotane; mitoxantrone; nandrolone phenpropionate; nofetumomab; oprelvekin; oxaliplatin; paclitaxel; pamidronate; pegademase; pegaspargase; pegfilgrastim; pentostatin; pentostatin; pipobroman; plicamycin; mithramycin; porfimer sodium; PP2; procarbazine; quinacrine; rasburicase; RC3095; rituximab; sargramostim; streptozocin; talc; tamoxifen; temozolomide; teniposide, VM-26; testolactone; thioguanine, 6-TG; thiotepa; topotecan; toremifene; tositumomab; trastuzumab; tretinoin, ATRA; UO126; uracil mustard; valrubicin; vinblastine; vincristine; vinorelbine; wortmanin and zoledronate.

84. The method of claim 82, wherein the anticancer agent is cisplatin.

85. The method of claim 82, wherein the anticancer agent is gossypol.

86. The method of claim 82, wherein the anticancer agent is selected from the group consisting of: tyrosine kinase inhibitors; antibodies or fragments thereof; angiogenesis inhibitors; immune modulators; non-tyrosine kinase inhibitors; serine/threonine kinase inhibitors; farnesyl or geranyl transferase inhibitors, such as FTI-277 and GGTI-298; and G-protein-coupled receptor inhibitors.

87. The method of claim 76, wherein the cell is a cell of a cancer selected from the group consisting of multiple myeloma; HTLV-1 dependent leukemia; acute myelogenous leukemia; large granular lymphocyte leukemia; lymphoma; EBV-related Burkitt's lymphoma; mycosis fungoides; cutaneous T-cell lymphoma; non-Hodgkins lymphoma; anaplastic large-cell lymphoma; breast cancer; melanoma; ovarian cancer; lung cancer; pancreatic cancer and prostate cancer.

88. A method of inducing apoptosis in a cancer cell of a patient in which STAT3 is activated, comprising administering to the patient an amount of the composition of claim 39 effective to induce apoptosis in the cancer cell in which STAT3-is activated, thereby inducing apoptosis in the cancer cell.

89. The method of claim 88, wherein the cancer cell is a cell of a squamous cell carcinoma.

90. The method of claim 88, wherein the cancer cell is a cell of a squamous cell carcinoma of the head and neck.

91. The method of claim 88, comprising administering to the patient a second anticancer therapy.

92. The method of claim 91, wherein the second anticancer therapy is one or both of a radiation therapy and treating the patient with an anticancer agent.

93. The method of claim 92, wherein the second anticancer therapy is a radiation therapy.

94. The method of claim 92, wherein the second anticancer therapy comprises treating the patient with an anticancer agent.

95. The method of claim 94, wherein the anticancer agent is selected from the group consisting of aldesleukin; alemtuzumab; alitretinoin; allopurinol; altretamine; amifostine; An-238; anastrozole; arsenic trioxide; asparaginase; BCG Live; bevacizumab; bexarotene; bleomycin; busulfan; calusterone; capecitabine; capecitabine; carboplatin; carmustine; celecoxib; cetuximab; chlorambucil; cisplatin; cladribine; cyclophosphamide; cyclophosphamide; cytarabine; dactinomycin; darbepoetin alfa; daunorubicin; daunorubicin, daunomycin; denileukin diftitox; dexrazoxane; docetaxel; doxorubicin; dromostanolone propionate; Elliott's B Solution; endostatin; epirubicin; epoetin alfa; estramustine; etoposide phosphate; etoposide, VP-16; exemestane; filgrastim; floxuridine; fludarabine; fluorouracil; FTI-277; fulvestrant; gefitinib; gemcitabine; gemcitabine; gemtuzumab ozogamicin; GGTI-298; goserelin acetate; hydroxyurea; ibritumomab; idarubicin; idarubicin; ifosfamide; imatinib mesylate; interferon alfa-2a; interferon alfa-2b; IL-2; IL-12; irinotecan; letrozole; leucovorin; levamisole; lomustine; meclorethamine; nitrogen mustard; megestrol acetate; melphalan, L-PAM; mercaptopurine, 6-MP; mesna; methotrexate; methoxsalen; mitomycin C; mitotane; mitoxantrone; nandrolone phenpropionate; nofetumomab; oprelvekin; oxaliplatin; paclitaxel; pamidronate; pegademase; pegaspargase; pegfilgrastim; pentostatin; pentostatin; pipobroman; plicamycin; mithramycin; porfimer sodium; PP2; procarbazine; quinacrine; rasburicase; RC3095; rituximab; sargramostim; streptozocin; talc; tamoxifen; temozolomide; teniposide, VM-26; testolactone; thioguanine, 6-TG; thiotepa; topotecan; toremifene; tositumomab; trastuzumab; tretinoin, ATRA; UO126; uracil mustard; valrubicin; vinblastine; vincristine; vinorelbine; wortmanin and zoledronate.

96. The method of claim 94, wherein the anticancer agent is cisplatin.

97. The method of claim 94, wherein the anticancer agent is gossypol.

98. The method of claim 94, wherein the anticancer agent is selected from the group consisting of: tyrosine kinase inhibitors; antibodies or fragments thereof; angiogenesis inhibitors; immune modulators; non-tyrosine kinase inhibitors; serine/threonine kinase inhibitors; famesyl or geranyl transferase inhibitors, such as FTI-277 and GGTI-298; and G-protein-coupled receptor inhibitors.

99. The method of claim 88, wherein the cancer cell is a cell of a cancer selected from the group consisting of multiple myeloma; HTLV-1 dependent leukemia; acute myelogenous leukemia; large granular lymphocyte leukemia; lymphoma; EBV-related Burkitt's lymphoma; mycosis fungoides; cutaneous T-cell lymphoma; non-Hodgkins lymphoma; anaplastic large-cell lymphoma; breast cancer; melanoma; ovarian cancer; lung cancer; pancreatic cancer and prostate cancer.

100. An article of manufacture comprising a package, a container within the package; one or more doses of a STAT3 decoy in a pharmaceutically acceptable carrier within the container; and a label or package insert providing an indication of the use for the one or more doses in treatment of a cancer comprising cells in which STAT3 is activated.

101. The article of claim 100, wherein the treatment of a cancer includes one or more of reducing growth of a cancer in which STAT3 is activated in a patient, interfering with STAT3 binding to a STAT3 response element in cancer cells of a patient in which STAT3 is activated and/or inducing apoptosis in cancer cells of a patient in which STAT3 is activated.

102. The article of claim 100, wherein the cancer is a cancer in which STAT3 is activated.

103. The article of claim 100, wherein the cancer is a squamous cell carcinoma.

104. The article of claim 100, wherein the cancer is a squamous cell carcinoma of the head and neck.

105. The article of claim 100, wherein the cancer is selected from the group consisting of multiple myeloma; HTLV-1 dependent leukemia; acute myelogenous leukemia; large granular lymphocyte leukemia; lymphoma; EBV-related Burkitt's lymphoma; mycosis fungoides; cutaneous T-cell lymphoma; non-Hodgkins lymphoma; anaplastic large-cell lymphoma; breast cancer; melanoma; ovarian cancer; lung cancer; pancreatic cancer and prostate cancer.

106. A method of decreasing expression of one or more genes under transcriptional control by one or more of a p53 response element, a gamma-interferon activated sequence and an Early Growth Response-1 transcription recognition sequence in a cell, comprising contacting the cell with a composition comprising an amount of a STAT3 decoy effective to decrease expression of the one or more genes subject to control by one or more of a p53 response element, a gamma-interferon activated sequence and an Early Growth Response-1 transcription recognition sequence in a cell, thereby decreasing expression of the one or more genes subject to control by a one or more of a p53 response element, a gamma-interferon activated sequence and an Early Growth Response-1 transcription recognition sequence in the cell.

107. The method of claim 106, wherein the one or more genes are one or more of a p53 gene, an Egr-1 gene and an allele or mutant of a p53 or Egr-1 gene.

108. The method of claim 106 wherein the cell is a cancer cell of a patient.

109. The method of claim 106, wherein the STAT3 decoy is a double-stranded DNA or an analog thereof comprising the STAT3 target sequence:
5′-(N₆)ₙ,-CAN₁TTCN₂CN₃TN₄AN₅TC-(N₇)ₘ-3′ (SEQ ID NO: 1),
wherein N₁, N₂, N₃, N₄ and N₅ are A, T, G or C, and one, two, three or all of the following conditions are met: N₁, is T; N₂ is C; N₃ is G, N₄ is A and N₅ is A, and N₆ and N₇ are A, T, G or C and n and m are independently 0-50.

110. The method of claim 109, the STAT3 decoy comprising a double-stranded DNA or an analog thereof comprising a derivative of the STAT3 target sequence:
(SEQ ID NO:2)
5′-(N₆)ₙ-CATTTCCCGTAAATC-(N₇)ₘ-3′,
or a sequence complementary thereto, in which N₆ and N₇ are A, T, G or C and n and m are independently 0-50, containing a single nucleotide insertion, deletion or substitution within the sequence 5′-CATTTCCCGTAAATC-3′ (SEQ ID NO: 2).


SEQUENCE LISTING (32 sequences)

SEQ ID NO: 1 - DNA, 15 bp, Artificial Sequence
  STAT3 Oligonucleotide sequence
  misc_feature (3)..(3): n is a, c, g, or t
  misc_feature (7)..(7): n is a, c, g, or t
  misc_feature (9)..(9): n is a, c, g, or t
  misc_feature (11)..(11): n is a, c, g, or t
  misc_feature (13)..(13): n is a, c, g, or t
canttcncntnantc

SEQ ID NO: 2 - DNA, 15 bp, Homo sapiens
catttcccgtaaatc

SEQ ID NO: 3 - DNA, 31 bp, Homo sapiens
agcttgtcgacatttcccgtaaatcgtcgag

SEQ ID NO: 4 - DNA, 15 bp, Artificial Sequence
  SIE oligonucleotide
cagttcccttaaatc

SEQ ID NO: 5 - DNA, 15 bp, Homo sapiens
gatttacgggaaatg

SEQ ID NO: 6 - DNA, 15 bp, Artificial Sequence
  STAT3 mutant oligonucleotide
catttcccttaaatc

SEQ ID NO: 7 - DNA, 15 bp, Artificial Sequence
  Complement to mutant control oligo
gatttaagggaaatg

SEQ ID NO: 8 - DNA, 31 bp, Artificial Sequence
  Complement to STAT3 oligo 2
ctcgacgatttacgggaaatgtcgacaagct

SEQ ID NO: 9 - DNA, 15 bp, Artificial Sequence
  Complement to SIE Oligo
gatttaagggaactg

SEQ ID NO: 10 - DNA, 15 bp, Artificial Sequence
  STAT3 Mutant 1 oligonucleotide
cagttcccgtaaatc

SEQ ID NO: 11 - DNA, 15 bp, Artificial Sequence
  Complement to Mutant 1 Oligo
gatttacgggaactg

SEQ ID NO: 12 - DNA, 15 bp, Artificial Sequence
  STAT3 mutant 2 oligo
catttcacgtaaatc

SEQ ID NO: 13 - DNA, 15 bp, Artificial Sequence
  Complement to Mutant 2 oligo
gatttaagggaaatg

SEQ ID NO: 14 - DNA, 15 bp, Artificial Sequence
  STAT3 Mutant 3 oligo
catttcccttaaatg

SEQ ID NO: 15 - DNA, 15 bp, Artificial Sequence
  Complement to Mutant 3 oligo
gatttaagggaaatg

SEQ ID NO: 16 - DNA, 15 bp, Artificial Sequence
  STAT3 Mutant 4 oligo
catttcccgtcaatc

SEQ ID NO: 17 - DNA, 15 bp, Artificial Sequence
  Complement to Mutant 4 oligo
gattgacgggaaatg

SEQ ID NO: 18 - DNA, 15 bp, Artificial Sequence
  STAT3 Mutant 5 oligo
cagttcacgtaaatc

SEQ ID NO: 19 - DNA, 15 bp, Artificial Sequence
  complement to Mutant 5 oligo
gatttacgtgaactg

SEQ ID NO: 20 - DNA, 15 bp, Artificial Sequence
  STAT3 Mutant 6 oligo
cagttcccgtcaatc

SEQ ID NO: 21 - DNA, 15 bp, Artificial Sequence
  Complement to Mutant 6 oligo
gattgacgggaactg

SEQ ID NO: 22 - DNA, 15 bp, Artificial Sequence
  STAT3 Mutant 7 oligo
catttcacgtcaatc

SEQ ID NO: 23 - DNA, 15 bp, Artificial Sequence
  Complement to Mutant 7 oligo
gattgaagggaaatg

SEQ ID NO: 24 - DNA, 15 bp, Artificial Sequence
  STAT3 Mutant 8 oligo
catttcccttcaatc

SEQ ID NO: 25 - DNA, 15 bp, Artificial Sequence
  Complement to Mutant 8 oligo
gattgaagggaaatg

SEQ ID NO: 26 - DNA, 15 bp, Artificial Sequence
  STAT3 Mutant 9 oligo
cagttcacgtcaatc

SEQ ID NO: 27 - DNA, 15 bp, Artificial Sequence
  Complement to Mutant 9 oligo
gattgacgtgaactg

SEQ ID NO: 28 - DNA, 15 bp, Artificial Sequence
  STAT3 Mutant 10 oligo
cagttcccttcaatc

SEQ ID NO: 29 - DNA, 15 bp, Artificial Sequence
  Complement to Mutant 10 oligo
gattgaagggaactg

SEQ ID NO: 30 - DNA, 17 bp, Homo sapiens
tgcctggacttgcctgg

SEQ ID NO: 31 - DNA, 21 bp, Homo sapiens
agtttcatattactctaaatc

SEQ ID NO: 32 - DNA, 9 bp, Homo sapiens
  misc_feature (9)..(9): n is a, c, g, or t
gggtggggn